(19) United States
(12) Patent Application Publication
(10) Pub. No.: US 2024/0423964 A1
(43) Pub. Date: Dec. 26, 2024

(54) METHODS AND COMPOSITIONS FOR IMPROVING NEUROMUSCULAR JUNCTION MORPHOLOGY AND FUNCTION

(71) Applicant: The Board of Trustees of the Leland Stanford Junior University, Stanford, CA (US)

(72) Inventors: Helen M. Blau, Stanford, CA (US); Yu Xin Wang, Stanford, CA (US); Mohsen Afshar Bakooshli, Stanford, CA (US)

(73) Assignee: The Board of Trustees of the Leland Stanford Junior University, Stanford, CA (US)

(21) Appl. No.: 18/701,357

(22) PCT Filed: Oct. 19, 2022
(86) PCT No.: PCT/US2022/078394

§ 371 Date: Apr. 15, 2024

Related U.S. Application Data
(60) Provisional application No. 63/257,264, filed on Oct. 19, 2021.

(51) Int. Cl. A61K 31/4365; A61K 31/426; A61P 25/00

(52) U.S. Cl. CPC A61K 31/4365; A61K 31/426; A61P 25/00; C12Y 101/01141

(57) ABSTRACT

The present disclosure provides methods of improving, enhancing, and/or rejuvernating neuromuscular junction morphology and/or function in a subject by administering to the subject an amount of a 15-PGDH inhibitor effective to inhibit 15-PGDH activity and/or reduce 15-PGDH levels in the subject. The methods described herein are useful for treating subjects afflicted with neurogenic myopathies, aged-induced loss of muscle mass, genetic neuromuscular wasting disorders, or after trauma or injury, among others.

### CROSS-REFERENCE TO RELATED APPLICATIONS

This application claims priority to U.S. Provisional Application No. 63/257,264, filed Oct. 19, 2021, the disclosure of which is hereby incorporated by reference in its entirety for all purposes.

### STATEMENT AS TO RIGHTS TO INVENTIONS MADE UNDER FEDERALLY SPONSORED RESEARCH AND DEVELOPMENT

This invention was made with Government support under contracts AG020961 and AG069858 awarded by the National Institutes of Health. The Government has certain rights in the invention.

### BACKGROUND

Denervation is a hallmark of age- and neurogenic-related myopathies. Neuromuscular junctions (NMJs) are specialized synapses between muscle fibers and the lower motor neurons of the spinal cord that innervate and control muscle locomotion. Acetylcholine receptors (AChRs) are major components of the NMJs. They organize in branched and network-like structures on the surface of muscle fibers (the post-synaptic compartment of the NMJ) and are responsible for transmission of signals from the nervous system to muscle. Several conditions such as aging, neuromuscular transmission disorders, disuse and muscular dystrophies lead to fragmentation of neuromuscular junctions. There are currently no therapeutic interventions that can improve, enhance, and/or rejuvenate the morphology and/or function of the NMJs and/or induce and/or promote formation of NMJs in a subject having degeneration of NMJs.

### BRIEF SUMMARY

There remains a need in the art for effective strategies to improve, enhance, and/or rejuvenate the morphology of the NMJ and/or function, and/or induce and/or promote formation of NMJs, for the treatment of conditions including neurogenic myopathies and aged-induced loss of muscle mass due to muscle denervation. The present disclosure satisfies this unmet need and provides other advantages as well.

In one aspect, the present disclosure provides a method of improving, enhancing, and/or rejuvenating neuromuscular junction (NMJ) morphology and/or function, and/or inducing and/or promoting formation of NMJs, in a subject having degeneration of NMJs, the method comprising: administering to the subject an amount of a 15-PGDH inhibitor effective to inhibit 15-PGDH activity and/or reduce 15-PGDH levels in the subject, thereby improving, enhancing, and/or rejuvenating NMJ morphology and/or function, and/or inducing and/or promoting formation of NMJs, in the subject.

In some embodiments, the method results in increased pre-synaptic motor neuron and postsynaptic acetylcholine receptor (AChR) juxtaposition and/or connectivity. In some embodiments, the method results in a decreased number of fragmented AChR clusters at the NMJ. In some embodiments, the method results in a decreased number of NMJs lacking the presence of motor neurons. In some embodiments, the method results in decreased blebbing of motor neuron axons. In some embodiments, the method results in decreased apoptosis of motor neurons. In some embodiments, the method results in enhanced NMJ morphology and/or increased functional conduction of nerve signals to the muscle.

In some embodiments, the method results in a decreased number of AChR-rich vesicles at the NMJ. In some embodiments, the method results in increased expression and/or localization of AChR at the NMJ. In some embodiments, the method results in decreased AChR degradation. In some embodiments, the method results in increased AChR stability.

In some embodiments, the method results in improved, enhanced, and/or rejuvenated mitochondrial morphology in motor neuron axon terminals at the NMJ. In some embodiments, the method results in improved, enhanced, and/or rejuvenated motor neuron synaptic terminals at the NMJ. In some embodiments, the method results in improved, enhanced, and/or rejuvenated skeletal muscle mass and/or neuromuscular function in the subject. In some embodiments, the subject has muscle denervation and/or partial muscle denervation.

In some embodiments, the subject has a neurogenic myopathy, an aged-induced loss of muscle mass, a genetic neuromuscular wasting disorder, nerve trauma or injury, muscle trauma or injury, or any combination thereof. In some embodiments, the genetic neuromuscular wasting disorder is spinal muscular atrophy (SMA), Duchenne muscular dystrophy (DMD), or amyotrophic lateral sclerosis (ALS). In some embodiments, the subject has or has experienced one or more selected from the group consisting of: acute peripheral nerve injury, muscle disuse, myopathy with neurogenic and autoimmune involvement with target fibers or tubular aggregate formation, and vascular myopathy.

In some embodiments, the acute peripheral nerve injury is selected from the group consisting of: contusion injury, compression-decompression injury, nerve cut, botulinum toxicity, injury due to tenotomy, and sports injury. In some embodiments, the compression-decompression injury is selected from the group consisting of: edema, carpal tunnel syndrome, Baker's cyst, and repetitive task injury. In some embodiments, the muscle disuse is selected from the group consisting of: immobilization after bone fracture, prolonged bed rest, recovery after surgery, recovery from ventilator, space flight, and sedentary life-style. In some embodiments, the myopathy with neurogenic and autoimmune involvement with target fibers or tubular aggregate formation is selected from the group consisting of Duchenne muscular dystrophy, Becker muscular dystrophy, limb girdle muscular dystrophy, central core disease, distal motor axonal neuropathy, multifocal motor neuropathy, amyotrophic lateral sclerosis, spinal muscular atrophy, multiple sclerosis, ataxia, myotonic dystrophy, neurogenic amyloidosis, proximal myopathy with tubular aggregates, rheumatoid arthritis, Sjögren's syndrome, and myasthenia gravis. In some embodiments, the myasthenia gravis is selected from the group consisting of: congenital myasthenia gravis, episodic myasthenia gravis, and Lambert-Eaton myasthenic syndrome.

In some embodiments, the 15-PGDH inhibitor is selected from the group consisting of a small molecule compound, a blocking antibody, a nanobody, and a peptide. In some embodiments, the 15-PGDH inhibitor is selected from the group consisting of: SW033291 and (+)-SW209415. In some embodiments, the 15-PGDH inhibitor is a thiazolidinedione analog with 15-PGDH inhibitory activity. In some embodiments, the 15-PGDH inhibitor is selected from the group consisting of an antisense oligonucleotide, microRNA, siRNA, and shRNA.

In some embodiments, the subject is a human. In some embodiments, the subject is less than 30 years of age. In some embodiments, the subject is at least 30 years of age. In some embodiments, the 15-PGDH inhibitor reduces or blocks 15-PGDH expression.

In some embodiments, the 15-PGDH inhibitor reduces or blocks enzymatic activity of 15-PGDH.

In some embodiments, the method is independent of muscle cell proliferation.

In some embodiments, the administering comprises systemic administration or local administration.

Other objects, features, and advantages of the present disclosure will be apparent to one of skill in the art from the following detailed description and figures.

### BRIEF DESCRIPTION OF THE DRAWINGS

FIGS. 1A-1D show that small molecule inhibition of 15-PGDH leads to improved aged muscle function by increasing muscle mass and force. FIG. 1A shows eicosanoid levels in muscle tissue lysates (n=8 young veh, n=9 young SW, n=8 aged veh, n=6 aged SW). FIG. 1B shows 15-PGDH specific enzymatic activity assayed in muscle tissues of vehicle and SW treated aged muscles normalized to vehicle treated (n=4 per age group). FIG. 1C shows gastrocnemius (GA, left) and Tibialis anterior (TA, right) weights in young and aged mice treated with vehicle or SW. FIG. 1D shows plantar flexion tetanic force (absolute values as torque).

FIGS. 2A-2D show that 15-PGDH inhibition for one month improves, enhances, and/or rejuvenates the NMJ morphology in aged mice. FIG. 2A shows Representative confocal images of AChRs counterstained with fluorophore conjugated bungarotoxin (BTX) in young (left), aged (31-months) vehicle treated (middle panel), and aged (31-months) SW-treated (right panel) mice. FIG. 2 B shows number of fragments per AChR cluster in neuromuscular junctions from EDL muscles. n=132, 138, and 217 NMJs for young, aged vehicle treated and aged SW-treated respectively. Statistical analysis was performed between experimental groups using one-way ANOVA followed by Tukey's multiple comparisons. FIG. 2C top panel shows maximum z-projection confocal images of AChR clusters from young (left panel), aged vehicle treated (middle panel), and aged SW-treated (right panel). FIG. 2C bottom panel shows AChR-rich vesicles are labeled with yellow circles. FIG. 2D shows bar graph quantification of BTX+ vesicles per AChR cluster. Minimum of 20 clusters were analyzed. n=20, 31, and 41 AChRs for young, aged vehicle, and aged SW-treated respectively. Statistical analysis was performed between experimental groups using one-way ANOVA followed by Tukey's multiple comparisons.

FIGS. 3A-3C show that 15-PGDH inhibition improves, enhances, and/or rejuvenates the morphology of MN associated mitochondria in distal axons and NMJs in aged mice. FIG. 3A shows representative electron micrographs of NMJs in young (left panel), aged vehicle (middle panel), and aged SW-treated (right panel). Axon terminals are outlined in yellow dashed circles. PNS: post-synaptic nuclei. FIG. 3B shows representative electron micrographs of distal axons in young (left panel), aged vehicle (middle panel), and aged SW-treated (right panel) showing the morphology and electron density of MN associated mitochondria. FIG. 3C shows quantification of mitochondria area in distal axons. Minimum of 12 axons from 2 animals were imaged and analyzed per condition.

FIGS. 4A-4F show that 15-PGDH gene expression is significantly increased in adult denervated muscle and 10 days old postnatal SMA mice. FIGS. 4A-4D show longitudinal analysis of muscle weight (FIG. 4A), 15-PGDH (FIG. 4B), MuRF1 (trim63) (FIG. 4C), and myostatin (FIG. 4D) mRNA expression in denervated gastrocnemius muscle in adult wild type mice. FIGS. 4E-4F show mRNA expression analysis of 15-PGDH (FIG. 4E), and myostatin (FIG. 4F) in tibialis anterior muscle of SMA A7 mice at post-natal day 10. Panels in FIGS. 4A-4D are a reanalysis of Ehmsen et al. (Sci Data, 6(1):179 (2019)), and panels in FIGS. 4E-4F are reanalysis of data published in McCormack et al. (J Cachexia Sarcopenia Muscle, 12(4):1098-1116 (2021)).

FIGS. 5A-5H show that 15-PGDH is upregulated in denervated muscle fibers. FIG. 5A depicts experimental scheme. FIG. 5B shows representative images of wholemount extensor digitorum longus (EDL) muscles immunostained for neurofilament (NF: red) and α-bungarotoxin (BTX: gray) in control (left panel) and denervated (right panel) legs 14 days post sciatic nerve transection. FIG. 5C shows representative CODEX immunofluorescence images of control (left panel) and denervated (right panel) tibialis anterior (TA) muscle cross-sections 14 days after sciatic nerve transection to co-detect 15-PGDH (yellow), cell membrane (WGA, blue), neurofilament (inset; NEFH, red), and alpha integrin (inset; a7-int, blue). Insets are magnified regions of white dashed squares in FIG. 5C highlighting TA nerve tracts. FIG. 5D shows representative western blot images of 15-PGDH in control (CTL) and denervated (DN) gastrocnemii of mice undergoing unilateral sciatic nerve transection 14 dpi. FIG. 5E shows bar graph quantification of 15-PGDH immunoblots normalized to total protein (n=5 mice, data are represented as mean±S.E.M). FIG. 5F shows kinetic measurement of 15-PGDH enzymatic activity in gastrocnemius muscle lysates from 4 mice control (black) and denervated (red) legs 14 days post unilateral sciatic nerve transection. FIG. 5G left panel shows representative chromatograph of 13,14-dihydro-15-keto PGE2 (PGEM) abundance analyzed by LC-MS/MS in control and denervated gastrocnemius muscles of young mice undergoing unilateral sciatic nerve transection. FIG. 5H right panel shows bar graph quantification of PGEM in gastrocnemius muscles quantified by LC-MS/MS (n=4 mice, data are represented as mean±S.E.M) 14 days post nerve transection. *P<0.05, **P<0.01.

FIGS. 6A-6E show 15-PGDH upregulation after denervation. FIG. 6A shows representative CODEX immunofluorescence images of control (left panel) and denervated (right panel) tibialis anterior muscle cross-sections 14 days post sciatic nerve transection. Smooth muscle actin (SMA; green) stains blood vessels, CD31 (red) stains endothelial cells, CD45 (gray) stains all immune cells, and DAPI (blue) counterstains cell nuclei. FIG. 6B shows CODEX images of control (top row) and denervated (bottom row) TA muscle cross-sections from 3 young mice undergoing unilateral sciatic nerve transection to visualize 15-PGDH 14 dpi. Images are pseudo-colored to represent 15-PGDH immunoreactivity in yellow. FIG. 6C shows loading control for 15-PGDH western blot presented in FIG. 5D as the Ponceau S staining. FIG. 6D shows representative chromatograph of PGE2 and PGD2 abundance analyzed by LC-MS/MS in control and denervated gastrocnemius muscles of young mice undergoing unilateral sciatic nerve transection. FIG. 6E shows bar graph quantification of PGE2 in gastrocnemius muscles quantified by LC-MS/MS (n=4 mice) 14 days post nerve transection. Data are presented at mean f S.E.M.

FIGS. 7A-7J show that 15-PGDH is part of a sustained autophagy response after denervation. FIG. 7A shows time course of normalized mRNA expression patterns of denervation associated genes CD11b (Itgam, red); MuRF1 (Trim63, orange); FoxO3 (green); 15-PGDH (Hpgd, blue); and NCAM1 (purple). Translucent error bands show the S.E.M. for each gene. FIG. 7B shows gene ontology terms enriched for each temporally distinct gene set (cluster) of denervation genes highlighted in FIG. 7A. FIG. 7C shows experimental scheme for single nuclei analysis of denervated GA muscles. FIG. 7D shows Annotations of cell types found in single nuclei analysis of denervated GA muscles plotted in an UMAP embedding. Each color denotes a cell type identified by clustering based on gene expression. FIG. 7E shows Kernel density estimation of the cell type composition for nuclei found in the contralateral leg (upper panel) versus the denervated leg (lower panel). FIG. 7F shows expression levels of 15-PGDH (Hpgd) detected in each nucleus. FIG. 7G shows gene ontology (GO) terms enriched for genes that positively correlate (r>0.5; upper set) or negatively correlate (r<−0.3; lower set) with 15-PGDH expression in myonuclei. FIG. 7H shows expression levels of LC3A (Map11c3a) detected in each nucleus. FIG. 7I shows expression levels of Parkin (Prkn) detected in each nucleus. FIG. 7J shows expression levels of VDAC1 (Vdac1; upper panel) and pyruvate dehydrogenase (Pdha1; lower panel) detected in each nucleus. Each dot denotes a nucleus in FIGS. 7D, 7F, 7H, 7J.

FIG. 8 shows temporally regulated genetic programs after denervation. Time course gene set enrichment analysis revealed 25 temporal dynamics (clusters) in differentially expressed genes after sciatic nerve transection (SNT). The top heatmap shows the mean normalized expression pattern of genes within each cluster across experimental time points (0, 1, 3, 7, 14, 21, 30, 90 days post SNT) in contralateral and denervated legs. The heatmap below shows gene ontology (biological processes) terms enriched for genes in each cluster. Top 10 terms ranked by Log 10 combined scores (log(P value)*(Z-score)) for each cluster are shown.

FIGS. 9A-9F show single nuclei analysis of skeletal muscle after denervation. FIG. 9A shows broad level annotations of cell types found in single nuclei analysis of denervated GA muscles plotted in an UMAP embedding. Each color denotes a cell type identified by clustering based on gene expression. FIG. 9B shows counts of analyzed nuclei that passed quality control for each broad cell type annotation shown in FIG. 9A. FIG. 9C shows UMAP embedding of analyzed nuclei showing the source of the GA muscle from either the contralateral (blue) or denervated (red) leg. FIG. 9D shows heatmap of the top 5 marker gene for each cell type cluster (from FIG. 7D). Marker genes are grouped by the cluster they are found in. Each row is a nuclei, ordered by cell type clusters. FIG. 9E shows Violin plot of 15-PGDH (Hpgd) expression in myonuclei subsets (myofiber types and specialized nuclei at the NMJ and MTJ). FIG. 9F shows ranked 15-PGDH correlated genes by Pearson correlation coefficient (r). Genes with r>0.5, corresponding to the inflection point of the curve, were considered positively correlated. Those with r<−0.3 were considered negatively correlated.

FIGS. 10A-10E show 15-PGDH aggregates in myofibers of aged mice and human neurogenic myopathies. FIG. 10A top panel shows representative CODEX immunofluorescence image of aged lateral gastrocnemius muscle to visualize myofiber subtypes (Type I: blue. Type IIa: green, Type IIb: red) and myofiber extracellular matrix (ECM) (laminin; gray). FIG. 10A bottom panel shows representative CODEX immunofluorescence image of the same region shown in the top panel stained to visualize 15-PGDH (yellow) and Dystrophin (DMD; blue). FIG. 10B shows quantification of 15-PGDH relative protein expression in young (gray bars) and aged (blue bars) soleus (slow-twitch), gastrocnemius (mixed fiber type), and extensor digitorum longus (fast-twitch) muscles (each dot represents an individual mouse, data are represented as mean±S.E.M). FIG. 10C shows representative immunofluorescence images of aged gastrocnemius muscle cross-section immunostained to visualize 15-PGDH (red), and autophagy marker LC3A (green). Cell nuclei are counter-stained with DAPI (blue). Myofibers are outlined in white dashed lines. FIG. 10D shows representative immunofluorescence images of aged gastrocnemius muscle cross-section immunostained to visualize 15-PGDH (red) and mitochondrial membrane ion channel VDAC1 (green). Myofibers basal lamina are counter-stained with wheat germ agglutinin (gray). Myofibers are outlined in white dashed lines. FIG. 10E shows representative images of muscle biopsy serial cross-sections from a patient diagnosed with a neurogenic myopathy immunostained to visualize left panel: 15-PGDH (red); middle panel: LC3A (green), 15-PGDH (red), WGA (ECM, gray), and DAPI (nuclei, blue); Right panel: mitochondrial membrane ion channel VDAC1 (green), mitochondrial enzyme pyruvate dehydrogenase PDHA (red), WGA (ECM, gray), and DAPI (nuclei, blue). ns: not significant. * P<0.05, ****P<0.0001.

FIGS. 11A-11F show 15-PGDH in fibers undergoing neurogenic remodeling. FIG. 11A shows representative immunofluorescence images of aged soleus (left panel) and extensor digitorum longus (EDL) (right panel) muscles immunostained to visualize myofiber subtypes (Type I: blue, Type IIa: gray, Type IIb: red) and myofiber basal lamina (laminin: green). FIG. 11B shows quantification of denervated myofibers (% total) in young (gray bars) and aged (blue bars) soleus and EDL muscles (each dot represents an individual mouse, data are represented as mean±S.E.M). FIG. 11C shows representative 15-PGDH immunoblots from young and aged soleus (top), gastrocnemius (middle), and extensor digitorum longus (bottom) muscle lysates. Ponceau S staining was used as the total protein loading control. FIG. 11D shows representative immunofluorescence image of neural cell adhesion molecule (NCAM, green) staining in aged gastrocnemius indicating a denervated myofiber. Myofiber basal lamina is stained with laminin (red). FIG. 11E shows representative immunofluorescence image of ubiquitin-binding protein p62 (a marker of autophagy, magenta) staining in an aged gastrocnemius myofiber. Myofiber sarcolemma is stained with dystrophin (yellow). FIG. 11F shows representative immunofluorescence image of ubiquitin staining (cyan) in an aged gastrocnemius myofiber. Myofiber basal lamina is stained with laminin (gray). ns: not significant. **P<0.01, ***P<0.001.

FIGS. 12A-12F show that 15-PGDH inhibition enhances recovery from nerve crush. FIG. 12A shows experimental scheme. FIG. 12B shows representative confocal images of neuromuscular junctions in the extensor digitorum longus muscles of injured legs of mice undergoing unilateral sciatic nerve transection 14 dpi. Mice were treated daily with SW or vehicle as control (veh) intraperitoneally as shown in FIG. 12A. Wholemount tissues were immunostained with a cocktail of antibodies to presynaptic motor neurons (neurofilament+synaptic vesicle, red) and fluorophore-conjugated α-bungarotoxin (BTX, green) to visualize postsynaptic AChRs. Nuclei are counterstained with Hoechst (blue). Arrowheads (white) indicate denervated NMJs lacking presynaptic (red) signal. FIG. 12C shows bar graph quantification of percent denervated myofibers in ipsilateral EDL muscles of mice undergoing unilateral sciatic nerve crush 14 dpi. Mice were treated daily intraperitoneally with either vehicle (blue) or SW (red) as shown in FIG. 12A. FIG. 12D shows plantar flexion tetanic force (percent of contralateral (control) leg) induced via nerve stimulation on days 3, 7, and 14 after nerve crush injury in vehicle (blue) and SW-treated (red) mice (n=4 each). FIG. 12E shows gastrocnemius (GA) and Soleus (Sol) weight in vehicle (blue) and SW (red) treated mice 14 days post nerve crush. FIG. 12F shows muscle specific force (plantar flexion tetanic force normalized to muscle weight) in vehicle (blue) and SW-treated (red) mice 14 days post nerve crush injury. dpi: days post injury, veh: vehicle, *P<0.05, **P<0.01, data in FIGS. 12C-12F are presented as mean±S.E.M.

FIGS. 13A-13E show that peripheral nerve injury increases spinal 15-PGDH activity. FIG. 13A shows stitched immunofluorescence image of lumbar spinal cord stained for IBA1 from mice undergoing unilateral sciatic nerve crush 14 days post-injury indicating accumulation of microglia around motor neurons in the ventral horn ipsilateral, but not contralateral, to the injury. Insets: magnified regions of areas outlined in yellow boxes. FIG. 13B left panel shows confocal image of sciatic nerve cross-section immunostained with Iba1 (green) and 15-PGDH (red). FIG. 13B right panels shows magnified regions from left indicating the co-localization of 15-PGDH staining in Iba1+microglial cells. FIG. 13C shows 15-PGDH immunoblots from spinal cord lysates of young untreated (control) or injured (14 days after undergoing sciatic nerve crush) mice. Gapdh was used as loading control. FIG. 13D shows densitometry of immunoreactive bands as in FIG. 13C. The ratio of 15-PGDH to Gapdh was determined and plotted as mean±SEM (n=4 each). FIG. 13E shows kinetic measurement of 15-PGDH specific activity in lumbar spinal cord of control and injured mice (n=4 each).

FIGS. 14A-14E show detailed characterization of muscle weight and function after nerve crush injury treated with 15-PGDH inhibition. FIG. 14A shows bar graph quantification of body weight of mice treated with vehicle (blue) and SW on days 0 and 14 post sciatic nerve crush surgery. FIG. 14B shows gastrocnemius (GA) and Soleus (Sol) weight of the contralateral uninjured legs (left legs) in vehicle (blue) and SW (red) treated mice 14 days post nerve crush (n=4 vehicle and n=3 SW). FIG. 14C shows plantar flexion tetanic force of the contralateral uninjured legs (left leg) induced via nerve stimulation on day 14 after nerve crush injury in vehicle (Veh, blue) and SW-treated (SW, red) mice (n=4 each, values are shown as % normalized to the average of vehicle-treated mice). FIGS. 14D-14E show bar graph quantification of plantar flexion tetanus force in control and injured legs of mice undergoing unilateral sciatic nerve crush 7 dpi (FIG. 14D) and 14 dpi (FIG. 14E). Blue bars represent vehicle-treated and red bars represent SW-treated mice. Solid bars represent control legs and diagonal patterns indicate injured legs. Data are presented as mean±S.E.M. in all bar graphs. dpi: days post injury, veh: vehicle, DN: denervated, ns: not significant, **P<0.01.

FIGS. 15A-15G show that 15-PGDH inhibition improves, enhances, and/or rejuvenates NMJs in aged mice. FIG. 15A shows experimental scheme. FIG. 15B shows representative confocal images of NMJs in EDL muscles of aged mice treated with vehicle (top panels) or SW (bottom panels). Wholemount muscle tissues were immunostained to visualized presynaptic motor neurons (red) and postsynaptic AChRs (green). Nuclei are counterstained in blue. FIGS. 15C-15E show bar graph quantification of age-related abnormalities (FIG. 15C: denervation, FIG. 15D: fragmentation, FIG. 15E: axonal swelling) in NMJs from EDL muscles of vehicle-treated (blue, n=7) and SW-treated (red, n=8) aged mice (>26 months). Each bar shows mean±S.E.M. FIG. 15F shows bar graph quantitative analysis of endo/lysosomal vesicles associated with AChRs in young (gray bar), aged vehicle-treated (blue), and aged SW-treated (red) mice. Each data point represents the average from one mouse. FIG. 15G shows representative oil-immersion confocal images of young and aged postsynaptic AChRs stained with α-bungarotoxin (BTX). Arrowheads indicate BTX-positive endo/lysosomal vesicles. Data are presented as mean f S.E.M in FIGS. 15C, 15C, 15D, 15E, and 15F. veh: vehicle, *P<0.05, ***P<0.001, ****P<0.0001.

FIGS. 16A-16D show that 15-PGDH inhibition is neuronal protective in aged mice. FIG. 16A Representative electron micrographs of heavily myelinated axons from longitudinal sections of extensor digitorum longus. (EDL) muscles of young, aged vehicle, and aged SW-treated mice illustrating motor neuron-associated mitochondria morphology. FIG. 16B shows quantification of mitochondria size in aged vehicle (blue) and SW (red) treated mice from transmission electron microscopy (TEM) images (n=5 mice per condition). FIG. 16C shows representative confocal images of ChAT+ neurons (red) in the ventral horn of the lumbar spinal cord co-immunostained with cleaved-caspase-3 (green). Nuclei are counterstained with Hoechst in blue. FIG. 16D shows Bar graph quantification of data presented in FIG. 16C. Percentage of ChAT+ cells in the ventral horn of lumbar spinal cord co-labeled with cleaved caspase 3. n=4 young vehicle, n=3 aged vehicle, and n=4 aged SW.

FIGS. 17A-17D show increased 15-PGDH activity in aged lumbar spinal cord. FIG. 17 A shows confocal images of ventral horns of lumbar spinal cords immunostained with Iba1 (microglia, green) and ChAT (motor neuron, red) from young (top panel) and aged (bottom panels) mice showing an increased incidence of activated microglia morphology in aged mice. FIGS. 17B-17C show quantification of microglia area and immunoreactivity (mean fluorescence intensity MFI) in young and aged lumbar spinal cord. Data are normalized to average young. FIG. 17D shows kinetic measurement of 15-PGDH specific activity in lumbar spinal cord of young and injured mice (n=4 each).

FIG. 18 shows examples of pathological NMJ morphology in aged mice. Oil-immersion confocal images of young and aged NMJs immunostained to visualize presynaptic motor neurons (red) and postsynaptic AChRs (green). Motor axons in aged NMJs show axon swellings (asterisks) while post-synaptic AChRs (green) show signs of fragmentation (arrowheads).

### DETAILED DESCRIPTION

### 1. Introduction

The skeletal muscle neuromuscular junction (NMJ) is a highly specialized synapse between a motor neuron axon and a muscle fiber. It transmits signals from the central nervous system to the muscle fibers in order to actuate fiber contraction. Acetylcholine receptors (AChRs), clustered at extremely high densities (10,000 molecules per square micron) at the NMJ post-synaptic muscle fiber, mediate this signal. Regardless of aging, denervation causes reduction of AChR stability. However, during aging, reduced stability of AChR is due to partly denervation and partly aged muscle fibers. During aging, different compartments of the NMJ, including the AChRs, undergo major morphological changes. For example, arising from partial muscle fiber denervation and neurogenic disturbances, AChR stability decreases with age. Partially denervated aged muscle fibers exhibit fragmented AChR structures unlike those found in young mice, and denervation upregulated genes such as Trim63 are also known to regulate AChR degradation in muscle fibers during aging.

There are currently no treatments to improve or enhance muscle mass and rejuvenate fragmented neuromuscular junctions and/or function, and/or induce and/or promote formation of NMJs in aged muscle that has experienced muscle denervation. Physical exercise and rehabilitation are the only therapeutic approaches to improve skeletal muscle innervation. While myostatin inhibitors have been proposed to improve, enhance, and/or rejuvenate muscle mass in dystrophic and sarcopenic muscles, direct myostatin inhibition therapies have failed in several clinical trials. The present disclosure demonstrates that inhibition of 15-hydroxyprostaglandin dehydrogenase (15-PGDH) can improve, enhance, and/or rejuvenate the neuromuscular junction (NMJ) morphology and/or function, and/or induce and/or promote formation of NMJs in a subject having degeneration of NMJs (e.g., in aged muscle or neurogenic myopathies (e.g., such as those that involve muscle denervation)). The disclosure herein shows that 15-PGDH gene and protein expression, and enzymatic activity are upregulated and persists for up to 90 days upon skeletal muscle denervation, making 15-PGDH an ideal molecular target to improve, enhance, and/or rejuvenate muscle mass in subjects that have experienced muscle denervation, and further distinguishes this method from inhibition of proteosome function, and/or inhibition of myostatin signaling as others have previously described. As such, the methods described herein are useful for improving, enhancing, and/or rejuvenating skeletal muscle mass and neuromuscular function in patients having degeneration of NMJs, e.g., patients who are afflicted with neurogenic myopathies, aged-induced loss of muscle mass (e.g., sarcopenia), genetic neuromuscular wasting disorders (e.g., spinal muscular atrophy (SMA), Duchenne muscular dystrophy (DMD), amyotrophic lateral sclerosis (ALS)), or after trauma or injury, among others.

### 2. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

“15-PGDH” (15-hydroxyprostaglandin dehydrogenase) is an enzyme involved in the inactivation of a number of active prostaglandins, e.g., by catalyzing oxidation of PGE2 to 15-keto-prostaglandin E2 (15-keto-PGE2), or the oxidation of PGD2 to 15-keto-prostaglandin D2 (15-keto-PGD2). The human enzyme is encoded by the HPGD gene (Gene ID: 3248). The enzyme is a member of the short-chain nonmetalloenzyme alcohol dehydrogenase protein family. Multiple isoforms of the enzyme exist, e.g., in humans, any of which can be targeted using the present methods. For example, any of human isoforms 1-6 (e.g., GenBank Accession Nos. NP_000851.2, NP_001139288.1. NP_001243236.1, NP_001243234.1, NP_001243235.1, NP 001350503.1, NP_001243230.1) can be targeted, as can any isoform with 50%, 60%, 70%, 80%, 85%, 90%, 95%, or higher identity to the amino acid sequences of any of GenBank Accession Nos. NP_000851.2, NP_001139288.1, NP_001243236.1, NP_001243234.1, NP 001243235.1, NP_001350503.1, NP_001243230.1, or of any other 15-PGDH enzyme.

A “15-PGDH inhibitor” refers to any agent that is capable of inhibiting, reducing, decreasing, attenuating, abolishing, eliminating, slowing, or counteracting in any way any aspect of the expression, stability, or activity of 15-PGDH. A 15-PGDH inhibitor can, for example, reduce any aspect of the expression, e.g., transcription, RNA processing, RNA stability, or translation of a gene encoding 15-PGDH, e.g., the human HPGD gene, by, e.g., 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. Similarly, a 15-PGDH inhibitor can, for example, reduce the activity, e.g., enzymatic activity, of a 15-PGDH enzyme by, e.g., 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. Further, a 15-PGDH inhibitor can, for example, reduce the stability of a 15-PGDH enzyme by, e.g., 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. A “15-PGDH inhibitor”, also referred to herein as an “agent” or a “compound,” can be any molecule, either naturally occurring or synthetic, e.g., peptide, protein, oligopeptide (e.g., from about 5 to about 25 amino acids in length, e.g., about 5, 10, 15, 20, or 25 amino acids in length), small molecule (e.g., an organic molecule having a molecular weight of less than about 2500 Daltons, e.g., less than 2000, less than 1000, or less than 500 Daltons), antibody, nanobody, polysaccharide, lipid, fatty acid, inhibitory RNA (e.g., siRNA, shRNA, microRNA), modified RNA, polynucleotide, oligonucleotide, e.g., antisense oligonucleotide, aptamer, affimer, drug compound, or other compound.

The phrase “specifically binds” refers to a molecule (e.g., a 15-PGDH inhibitor such as a small molecule or antibody) that binds to a target with greater affinity, avidity, more readily, and/or with greater duration to that target in a sample than it binds to a non-target compound. In some embodiments, a molecule that specifically binds a target (e.g., 15-PGDH) binds to the target with at least 2-fold greater affinity than non-target compounds, e.g., at least 3-fold, 4-fold, 5-fold, 6-fold, 7-fold, 8-fold, 9-fold, 10-fold, 20-fold, 25-fold, 50-fold or greater affinity. For example, in some embodiments, a molecule that specifically binds to 15-PGDH, will typically bind to 15-PGDH with at least a 2-fold greater affinity than to a non-15-PGDH target.

The term “derivative,” in the context of a compound, includes but is not limited to, amide, ether, ester, amino, carboxyl, acetyl, and/or alcohol derivatives of a given compound.

The term “treating” or “treatment” refers to any one of the following: ameliorating one or more symptoms of a disease or condition; preventing the manifestation of such symptoms before they occur, slowing down or completely preventing the progression of the disease or condition (as may be evident by longer periods between reoccurrence episodes, slowing down or prevention of the deterioration of symptoms, etc.); enhancing the onset of a remission period; slowing down the irreversible damage caused in the progressive-chronic stage of the disease or condition (both in the primary and secondary stages); delaying the onset of said progressive stage; or any combination thereof.

The term “administer,” “administering,” or “administration” refers to the methods that may be used to enable delivery of agents or compositions such as the compounds described herein to a desired site of biological action. These methods include, but are not limited to, parenteral administration (e.g., intravenous, subcutaneous, intraperitoneal, intramuscular, intra-arterial, intravascular, intracardiac, intrathecal, intranasal, intradermal, intravitreal, and the like), transmucosal injection, oral administration, administration as a suppository, and topical administration. One skilled in the art will know of additional methods for administering a therapeutically effective amount of the compounds described herein for preventing or relieving one or more symptoms associated with a disease or condition.

The term “therapeutically effective amount” or “therapeutically effective dose” or “effective amount” refers to an amount of a compound (e.g., 15-PGDH inhibitor) that is sufficient to bring about a beneficial or desired clinical effect. A therapeutically effective amount or dose may be based on factors individual to each patient, including, but not limited to, the patient's age, size, type or extent of disease or condition, stage of the disease or condition, route of administration, the type or extent of supplemental therapy used, ongoing disease process and type of treatment desired (e.g., aggressive vs. conventional treatment). Therapeutically effective amounts of a pharmaceutical compound or composition, as described herein, can be estimated initially from cell culture and animal models. For example, IC₅₀ values determined in cell culture methods can serve as a starting point in animal models, while IC₅₀ values determined in animal models can be used to find a therapeutically effective dose in humans.

The term “pharmaceutically acceptable carrier” refers to a carrier or a diluent that does not cause significant irritation to an organism and does not abrogate the biological activity and properties of the administered compound.

The terms “subject,” “individual,” and “patient” are used interchangeably herein to refer to a vertebrate, preferably a mammal, more preferably a human. Mammals include, but are not limited to, murines, rats, simians, humans, farm animals or livestock for human consumption such as pigs, cattle, and ovines, as well as sport animals and pets. Subjects also include vertebrates such as fish and poultry.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs. SNPs, and complementary sequences as well as the sequence explicitly indicated. In particular embodiments, modified RNA molecules are used, e.g., mRNA with certain chemical modifications to allow increased stability and/or translation when introduced into cells, as described in more detail below. It will be appreciated that any of the RNAs used in the present methods, including nucleic acid inhibitors such as siRNA or shRNA, can be used with chemical modifications to enhance, e.g., stability and/or potency, e.g., as described in Dar et al., Scientific Reports 6: article no. 20031 (2016), and as presented in the database accessible at crdd.osdd.net/servers/sirnamod/.

“Polypeptide”, “peptide”, and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

### 3. 15-PGDH Inhibitors

Any agent that reduces, decreases, counteracts, attenuates, inhibits, blocks, downregulates, or eliminates in any way the expression, stability or activity, e.g., enzymatic activity, of 15-PGDH can be used in the present methods. Inhibitors can be small molecule compounds, peptides, polypeptides, nucleic acids, antibodies, e.g., blocking antibodies or nanobodies, or any other molecule that reduces, decreases, counteracts, attenuates, inhibits, blocks, downregulates, or eliminates in any way the expression, stability, and/or activity of 15-PGDH, e.g., the enzymatic activity of 15-PGDH. In some embodiments, the 15-PGDH inhibitor comprises a small molecule compound, a blocking antibody, a nanobody, and a peptide. In some embodiments, the 15-PGDH inhibitor is SW033291, and/or (+)-SW209415, and/or thiazolidinedione analogues with 15-PGDH inhibitory activity (e.g., such as any 15-PGDH inhibitor described in “Synthesis and Biological Evaluation of Novel Thiazolidinedione Analogues as 15-Hydroxyprostaglandin Dehydrogenase Inhibitors”, Wu et al., J. Med. Chem. 2011, 54, 14, 5260-5264; which is herein incorporated by reference with respect to its disclosure of 15-PGDH inhibitors). In some embodiments, the 15-PGDH inhibitor is selected from the group consisting of an antisense oligonucleotide, microRNA, siRNA, or shRNA.

In some embodiments, the inhibition of 15-PDGH protein and/or activity improves PGE2 level to the level similar to young mice. In some embodiments, the inhibition of 15-PDGH protein and/or activity increases plantar flexion force. In some embodiments, the inhibition of 15-PDGH protein and/or activity promote muscle mass. In some embodiments, the inhibition of 15-PDGH protein and/or activity accelerates recovery and improve, enhance, or rejuvenate motor neuron function after peripheral nerve injury.

In some embodiments, the inhibition of 15-PDGH protein and/or activity induce and/or promote formation of NMJs. In some embodiments, the inhibition of 15-PDGH protein and/or activity promote muscle-neuronal connectivity. In some embodiments, the inhibition 15-PDGH protein and/or activity increases number of mitochondria and/or improve mitochondria morphology in neurons. In some embodiments, the inhibition 15-PDGH protein and/or activity reduces denervation in nerve injury. In some embodiments, the inhibition 15-PDGH protein and/or activity reduces apoptosis markers. e.g., cleaved caspase-3, in neurons.

In some embodiments, the inhibition of 15-PDGH protein and/or activity improves, enhances, and/or rejuvenate morphology and/or function of neuro-muscular junction (NMJ). In some embodiments, the improvement of morphology of NMJ comprises an improved morphology of acetylcholine receptor (AChR), which is described as intact morphology and not fragmented. In some embodiments, the improvement of morphology of NMJ comprises improving, enhancing, and/or rejuvenating of AChR stability in postsynaptic fibers. In some embodiments, the improvement of morphology of NMJ comprises reducing number of AChR rich vesicles, e.g., endosome vesicles and/or lysosome vesicles. In some embodiments, the improvement of morphology of NMJ comprises improving, enhancing, and/or rejuvenating morphology of mitochondria in neurons. In some embodiments, the neurons comprise motor neuron. In some embodiments, the improvement of morphology of NMJ is examined using electromicroscopy analysis. In some embodiments, an axon terminal of the motor neurons is examined to investigate the improvement of morphology of NMJ.

In some embodiments, the inhibition of 15-PDGH protein and/or activity results in a decreased number of fragmented acetylcholine receptor (AChR) clusters at the NMJ. In some embodiments, the inhibition of 15-PDGH protein and/or activity results in decreased number of NMJs lacking the presence of motor neurons. In some embodiments, the inhibition of 15-PDGH protein and/or activity results in decreased blebbing of motor neuron axons. In some embodiments, the inhibition of 15-PDGH protein and/or activity results in decreased apoptosis of motor neurons. In some embodiments, the inhibition of 15-PDGH protein and/or activity results in enhanced NMJ morphology (e.g., as determined by interconnected AChR morphology (e.g., pretzel-shaped), on the muscle fiber). In some embodiments, the inhibition of 15-PDGH protein and/or activity results in a decreased number of AChR-rich vesicles at the NMJ. In some embodiments, the inhibition of 15-PDGH protein and/or activity result in increased expression and/or localization of AChR at the NMJ. In some embodiments, the inhibition of 15-PDGH protein and/or activity results in decreased AChR degradation. In some embodiments, the inhibition of 15-PDGH protein and/or activity results in increased AChR stability. In some embodiments, the inhibition of 15-PDGH protein and/or activity results in improved, enhanced, and/or rejuvenated mitochondrial morphology in motor neuron axon terminals at the NMJ. In some embodiments, the inhibition of 15-PDGH protein and/or activity results in improved, enhanced, and/or rejuvenated motor neuron synaptic terminals at the NMJ. In some embodiments, the inhibition of 15-PDGH protein and/or activity results in improved, enhanced, and/or rejuvenated skeletal muscle mass and/or neuromuscular function in the subject.

In some embodiments, the inhibition of 15-PDGH protein and/or activity reduces catabolic regulators of muscle atrophic markers, e.g., atrogenes, ubiquitin ligases MuRF1 (Trim63) and Fbxo32 (Atrogene-1), and TGF-beta signaling. In some embodiments, the inhibition of 15-PDGH protein and/or activity reduces autophagy and mitophagy. In some embodiments, the inhibition of 15-PDGH protein and/or activity reduces autophagic marker. e.g., LC3A (Map11c3a), autophagosome marker p62, and ubiquitin, and/or mitophagic marker, e.g., parkin (Prkn). In some embodiments, the inhibition of 15-PDGH protein and/or activity improves, enhances, and/or rejuvenate mitochondria morphology and/or number.

In some embodiments, the inhibition of 15-PDGH protein and/or activity modulates, and/or increase, and/or decrease the expression of genes associated with peptidyl-lysine deacetylation, e.g., Hdac4, apoptosis, e.g., anoikis, or NMJ development. In some embodiments, the inhibition of 15-PDGH protein and/or activity improve and/or induce genes associated with mitochondrial activity or oxidative phosphorylation. In some embodiments, the inhibition of 15-PDGH protein and/or activity reduces genes associated with p53 signaling, cellular senescence, FoxO signaling, TGFbeta signaling, and/or autophagy. In some embodiments, the inhibition of 15-PDGH protein and/or activity reduces denervation marker NCAM1. In some embodiments, the inhibition of 15-PDGH protein and/or activity increases expression of mitochondrial membrane marker, e.g., voltage-dependent anion channel (VDAC1), and mitochondrial glycolytic enzyme pyruvate dehydrogenase (PDHA1).

In some embodiments, the inhibition of 15-PDGH protein and/or activity has an indirect effect on NMJ cells comprising neurons, and/or Schwann cells, and/or microglia, and/or muscle fibers.

In some embodiments, the inhibition of 15-PDGH protein and/or activity ameliorates abnormalities in motor neuron synapses at the NMJ.

In some embodiments, the 15-PGDH inhibitor decreases the activity, stability, or expression of 15-PGDH by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or more relative to a control level, e.g., in the absence of the inhibitor, in vivo or in vitro.

The efficacy of inhibitors can be assessed, e.g., by measuring 15-PGDH enzyme activity. e.g., using standard methods such as incubating a candidate compound in the presence of 15-PGDH enzyme, NAD(+), and PGE2 in an appropriate reaction buffer, and monitoring the generation of NADH (see, e.g., Zhang et al., (2015) Science 348: 1224), or by using any of a number of available kits such as the fluorometric PicoProbe 15-PGDH Activity Assay Kit (BioVision), or by using any of the methods and/or indices described in, e.g., EP 2838533 B1.

The efficacy of inhibitors can also be assessed, e.g., by detection of decreased polynucleotide (e.g., mRNA) expression, which can be analyzed using routine techniques such as RT-PCR, Real-Time RT-PCR, semi-quantitative RT-PCR, quantitative polymerase chain reaction (qPCR), quantitative RT-PCR (qRT-PCR), multiplexed branched DNA (bDNA) assay, microarray hybridization, or sequence analysis (e.g., RNA sequencing (“RNA-Seq”)). Methods of quantifying polynucleotide expression are described, e.g., in Fassbinder-Orth, Integrative and Comparative Biology, 2014, 54:396-406: Thellin et al., Biotechnology Advances. 2009, 27:323-333; and Zheng et al., Clinical Chemistry, 2006, 52:7 (doi: 10/1373/clinchem.2005.065078). In some embodiments, real-time or quantitative PCR or RT-PCR is used to measure the level of a polynucleotide (e.g., mRNA) in a biological sample. See. e.g., Nolan et al., Nat. Protoc, 2006, 1:1559-1582; Wong et al., BioTechniques, 2005, 39:75-75. Quantitative PCR and RT-PCR assays for measuring gene expression are also commercially available (e.g., TaqMan Gene Expression Assays, ThermoFisher Scientific). In some embodiments, the 15-PGDH inhibitor reduces or blocks 15-PGDH expression. In some embodiments, the 15-PGDH inhibitor reduces or blocks enzymatic activity of 15-PGDH.

In some embodiments, the 15-PGDH inhibitor is considered effective if the level of expression of a 15-PGDH-encoding polynucleotide is decreased by at least 10%, at least 20%, at least 30%, at least 40%, at least 50%, at least 60%, at least 70%, at least 80%, at least 90% or more as compared to the reference value, e.g., the value in the absence of the inhibitor, in vitro or in vivo. In some embodiments, a 15-PGDH inhibitor is considered effective if the level of expression of a 15-PGDH-encoding polynucleotide is decreased by at least 1.5-fold, at least 2-fold, at least 3-fold, at least 4-fold, at least 5-fold, at least 6-fold, at least 7-fold, at least 8-fold, at least 9-fold, at least 10-fold or more as compared to the reference value.

The effectiveness of a 15-PGDH inhibitor can also be assessed by detecting protein expression or stability, e.g., using routine techniques such as immunoassays, two-dimensional gel electrophoresis, and quantitative mass spectrometry that are known to those skilled in the art. Protein quantification techniques are generally described in “Strategies for Protein Quantitation,” Principles of Proteomics, 2nd Edition, R. Twyman, ed., Garland Science, 2013. In some embodiments, protein expression or stability is detected by immunoassay, such as but not limited to enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); immunofluorescence (IF); fluorescence polarization immunoassays (FPIA); and chemiluminescence assays (CL). If desired, such immunoassays can be automated. Immunoassays can also be used in conjunction with laser induced fluorescence (see. e.g., Schmalzing et al., Electrophoresis, 18:2184-93 (1997); Bao, J. Chromatogr. B. Biomed. Sci., 699:463-80 (1997)).

For determining whether 15-PGDH protein levels are decreased in the presence of a 15-PGDH inhibitor, the method comprises comparing the level of the protein (e.g., 15-PGDH protein) in the presence of the inhibitor to a reference value, e.g., the level in the absence of the inhibitor. In some embodiments, a 15-PGDH protein is decreased in the presence of an inhibitor if the level of the 15-PGDH protein is decreased by at least 10%, at least 20%, at least 30%, at least 40%, at least 50%, at least 60%, at least 70%, at least 80%, at least 90% or more as compared to the reference value. In some embodiments, a 15-PGDH protein is decreased in the presence of an inhibitor if the level of the 15-PGDH protein is decreased by at least 1.5-fold, at least 2-fold, at least 3-fold, at least 4-fold, at least 5-fold, at least 6-fold, at least 7-fold, at least 8-fold, at least 9-fold, at least 10-fold or more as compared to the reference value.

### Small Molecules

In particular embodiments, 15-PGDH is inhibited by the administration of a small molecule inhibitor. Any small molecule inhibitor can be used that reduces, e.g., by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or more, the expression, stability, or activity of 15-PGDH relative to a control, e.g., the expression, stability, or activity in the absence of the inhibitor. In particular embodiments, small molecule inhibitors may be used that can reduce the enzymatic activity of 15-PGDH in vitro or in vivo. Non-limiting examples of small molecule compounds that can be used in the present methods include the small molecules disclosed in EP 2838533 B1, the entire disclosure of which is herein incorporated by reference. Small molecules can include, inter alia, the small molecules disclosed in Table 2 of EP 2838533 B1, i.e., SW033291, SW033291 isomer B, SW033291 isomer A, SW033292, 413423, 980653, 405320, SW208078, SW208079, SW033290, SW208080, SW208081, SW206976, SW206977, SW206978, SW206979, SW206980, SW206992, SW208064, SW208065, SW208066, SW208067, SW208068, SW208069, SW208070, as well as combinations, derivatives, isomers, or tautomers thereof. In particular embodiments, the 15-PGDH inhibitor used is SW033291 (2-(butylsulfinyl)-4-phenyl-6-(thiophen-2-yl)thieno[2,3-b]pyridin-3-amine; PubChem CID: 3337839).

In some embodiments, the 15-PGDH inhibitor is a thiazolidinedione derivative (e.g., benzylidenethiazolidine-2,4-dione derivative) such as (5-(4-(2-(thiophen-2-yl)ethoxy)benzylidene)thiazolidine-2,4-dione), 5-(3-chloro-4-phenylethoxybenzylidene)thiazolidine-2,4-dione, 5-(4(2-cyclohexylethoxy)benzylidene)thiazolidine-2,4-dione, 5-(3-chloro-4-(2-cyclohexylethoxy)benzyl)thiazolidine-2,4-dione, (Z)-N-benzyl-4-((2,4-dioxothiazolidin-5-ylidene)methyl)benzamide, or any of the compounds disclosed in Choi et al. (2013) Bioorganic & Medicinal Chemistry 21:4477-4484; Wu et al. (2010) Bioorg. Med Chem. 18(2010) 1428-1433; Wu et al. (2011) J Med. Chem. 54:5260-5264; or Yu et al. (2019) Biotechnology and Bioprocess Engineering 24:464-475, the entire disclosures of which are herein incorporated by reference. In some embodiments, the 15-PGDH inhibitor is a COX inhibitor or chemopreventive agent such as ciglitazone (CID: 2750), or any of the compounds disclosed in Cho et al. (2002) Prostaglandins, Leukotrienes and Essential Fatty Acids 67(6):461-465, the entire disclosure of which is herein incorporated by reference.

In some embodiments, the 15-PGDH inhibitor is a compound containing a benzimidazole group, such as (1-(4-methoxyphenyl)-1H-benzo[d]imidazol-5-yl)(piperidin-1-yl)methanone (CID: 3474778), or a compound containing a triazole group, such as 3-(2,5-dimethyl-1-(p-tolyl)-1H-pyrrol-3-yl)-6,7,8,9-tetrahydro-5H-[1,2,4]triazolo[4,3-a]azepine (CID: 71307851), or any of the compounds disclosed in Duveau et al. (2015) (“Discovery of two small molecule inhibitors, ML387 and ML388, of human NAD+-dependent 15-hydroxyprostaglandin dehydrogenase,” published in Probe Reports from the NIH Molecular Libraries Program [Internet]), the entire disclosure of which is herein incorporated by reference. In some embodiments, the 15-PGDH inhibitor is 1-(3-methylphenyl)-1H-benzimidazol-5-yl)(piperidin-1-yl)methanone (CID: 4249877) or any of the compounds disclosed in Niesen et al. (2010) PLoS ONE 5(11):e13719, the entire disclosure of which is herein incorporated by reference. In some embodiments, the 15-PGDH inhibitor is 2-((6-bromo-4H-imidazo[4,5-b]pyridin-2-ylthio)methyl)benzonitrile (CID: 3245059), piperidin-1-yl(l-m-tolyl-1H-benzo[d]imidazol-5-yl)methanone (CID: 3243760), or 3-(2,5-dimethyl-1-phenyl-1H-pyrrol-3-yl)-6,7,8,9-tetrahydro-5H-[1,2,4]triazolo[4,3-a]azepine (CID: 2331284), or any of the compounds disclosed in Jadhav et al. (2011) (“Potent and selective inhibitors of NAD+-dependent 15-hydroxyprostaglandin dehydrogenase (HPGD),” published in Probe Reports from the NIH Molecular libraries Program [Internet]), the entire disclosure of which is herein incorporated by reference.

In some embodiments, the 15-PGDH inhibitor is TD88 or any of the compounds disclosed in Seo et al. (2015) Prostaglandins, Leukotrienes and Essential Fatty Acids 97:35-41, or Shao et al. (2015) Genes & Diseases 2(4):295-298, the entire disclosures of which are herein incorporated by reference. In some embodiments, the 15-PGDH inhibitor is EEAH (Ethanol extract of Artocarpus heterophyllus) or any of the compounds disclosed in Kama (2017) Pharmacogn Mag. 2017 January; 13(Suppl 1): S122-S126, the entire disclosure of which is herein incorporated by reference.

### Inhibitory Nucleic Acids

In some embodiments, the agent comprises an inhibitory nucleic acid, e.g., antisense DNA or RNA, small interfering RNA (siRNA), microRNA (miRNA), or short hairpin RNA (shRNA). In some embodiments, the inhibitory RNA targets a sequence that is identical or substantially identical (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical) to a target sequence in a 15-PGDH polynucleotide (e.g., a portion comprising at least 20, at least 30, at least 40, at least 50, at least 60, at least 70, at least 80, at least 90, or at least 100 contiguous nucleotides, e.g., from 20-500, 20-250, 20-100, 50-500, or 50-250 contiguous nucleotides of a 15-PGDH-encoding polynucleotide sequence (e.g., the human HPGD gene, Gene ID: 3248, including of any of its transcript variants, e.g., as set forth in GenBank Accession Nos. NM_000860.6, NM_001145816.2, NM_001256301.1, NM_001256305.1. NM_001256306.1. NM_001256307.1, or NM_001363574.1).

In some embodiments, the methods described herein comprise treating a subject, e.g., a subject having a neurogenic myopathy, an aged-induced loss of muscle mass, a genetic neuromuscular wasting disorder, or muscle trauma or injury, using an shRNA or siRNA. A shRNA is an artificial RNA molecule with a hairpin turn that can be used to silence target gene expression via the siRNA it produces in cells. &ee. e.g., Fire et. al., Nature 391:806-811, 1998; Elbashir et al., Nature 411:494-498, 2001; Chakraborty et al., Mol Ther Nucleic Acids 8:132-143, 2017; and Bouard et al., Br. J. Pharmacol. 157:153-165, 2009. In some embodiments, a method of treating a subject, e.g., having a neurogenic myopathy, an aged-induced loss of muscle mass, a genetic neuromuscular wasting disorder, or muscle trauma or injury, comprises administering to the subject a therapeutically effective amount of a modified RNA or a vector comprising a polynucleotide that encodes an shRNA or siRNA capable of hybridizing to a portion of a 15-PGDH mRNA (e.g., a portion of the human 15-PGDH-encoding polynucleotide sequence set forth in any of GenBank Accession Nos. NM_000860.6, NM_001145816.2, NM_001256301.1, NM_001256305.1, NM_001256306.1, NM_001256307.1, or NM_001363574.1). In some embodiments, the vector further comprises appropriate expression control elements known in the art, including, e.g., promoters (e.g., inducible promoters or tissue specific promoters), enhancers, and transcription terminators.

In some embodiments, the agent is a 15-PGDH-specific microRNA (miRNA or miR). A microRNA is a small non-coding RNA molecule that functions in RNA silencing and post-transcriptional regulation of gene expression. miRNAs base pair with complementary sequences within the mRNA transcript. As a result, the mRNA transcript may be silenced by one or more of the mechanisms such as cleavage of the mRNA strand, destabilization of the mRNA through shortening of its poly(A) tail, and decrease in the translation efficiency of the mRNA transcript into proteins by ribosomes.

In some embodiments, the agent may be an antisense oligonucleotide, e.g., an RNase H-dependent antisense oligonucleotide (ASO). ASOs are single-stranded, chemically modified oligonucleotides that bind to complementary sequences in target mRNAs and reduce gene expression both by RNase H-mediated cleavage of the target RNA and by inhibition of translation by steric blockade of ribosomes. In some embodiments, the oligonucleotide is capable of hybridizing to a portion of a 15-PGDH mRNA (e.g., a portion of a human 15-PGDH-encoding polynucleotide sequence as set forth in any of GenBank Accession Nos. NM_000860.6, NM_001145816.2, NM_001256301.1, NM_001256305.1, NM_001256306.1, NM_001256307.1, or NM_001363574.1). In some embodiments, the oligonucleotide has a length of about 10-30 nucleotides (e.g., 10, 12, 14, 16, 18, 20, 22, 24, 26, 28, or 30 nucleotides). In some embodiments, the oligonucleotide has 100% complementarity to the portion of the mRNA transcript it binds. In other embodiments, the DNA oligonucleotide has less than 100% complementarity (e.g., 95%, 90%, 85%, 80%, 75%, or 70% complementarity) to the portion of the mRNA transcript it binds, but can still form a stable RNA:DNA duplex for the RNase H to cleave the mRNA transcript.

Suitable antisense molecules, siRNA, miRNA, and shRNA can be produced by standard methods of oligonucleotide synthesis or by ordering such molecules from a contract research organization or supplier by providing the polynucleotide sequence being targeted. The manufacture and deployment of such antisense molecules in general terms may be accomplished using standard techniques described in contemporary reference texts: for example, Gene and Cell Therapy: Therapeutic Mechanisms and Strategies. 4^{th} edition by N. S. Templeton; Translating Gene Therapy to the Clinic: Techniques and Approaches. 1^{st} edition by J. Laurence and M. Franklin; High-Throughput RNAi Screening: Methods and Protocols (Methods in Molecular Biology) by D. O. Azorsa and S. Arora and Oligonucleotide-Based Drugs and Therapeutics: Preclinical and Clinical Considerations by N. Ferrari and R. Segui.

Inhibitory nucleic acids can also include RNA aptamers, which are short, synthetic oligonucleotide sequences that bind to proteins (see. e.g., Li et al., Nuc. Acids Res. (2006), 34:6416-24). They are notable for both high affinity and specificity for the targeted molecule, and have the additional advantage of being smaller than antibodies (usually less than 6 kD). RNA aptamers with a desired specificity are generally selected from a combinatorial library, and can be modified to reduce vulnerability to ribonucleases, using methods known in the art.

### Antibodies

In some embodiments, the agent is an anti-15-PGDH antibody or an antigen-binding fragment thereof. In some embodiments, the antibody is a blocking antibody (e.g., an antibody that binds to a target and directly interferes with the target's function, e.g., 15-PGDH enzyme activity). In some embodiments, the antibody is a neutralizing antibody (e.g., an antibody that binds to a target and negates the downstream cellular effects of the target). In some embodiments, the antibody binds to human 15-PGDH.

In some embodiments, the antibody is a monoclonal antibody. In some embodiments, the antibody is a polyclonal antibody. In some embodiments, the antibody is a chimeric antibody. In some embodiments, the antibody is a humanized antibody. In some embodiments, the antibody is a human antibody. In some embodiments, the antibody is an antigen-binding fragment, such as a F(ab′)2, Fab′, Fab, scFv, and the like. The term “antibody or antigen-binding fragment” can also encompass multi-specific and hybrid antibodies, with dual or multiple antigen or epitope specificities.

In some embodiments, an anti-15-PGDH antibody comprises a heavy chain sequence or a portion thereof, and/or a light chain sequence or a portion thereof, of an antibody sequence disclosed herein. In some embodiments, an anti-15-PGDH antibody comprises one or more complementarity determining regions (CDRs) of an anti-15-PGDH antibody as disclosed herein. In some embodiments, an anti-15-PGDH antibody is a nanobody, or single-domain antibody (sdAb), comprising a single monomeric variable antibody domain, e.g., a single VHH domain.

For preparing an antibody that binds to 15-PGDH, many techniques known in the art can be used. See, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2nd ed. 1986)). In some embodiments, antibodies are prepared by immunizing an animal or animals (such as mice, rabbits, or rats) with an antigen for the induction of an antibody response. In some embodiments, the antigen is administered in conjugation with an adjuvant (e.g., Freund's adjuvant). In some embodiments, after the initial immunization, one or more subsequent booster injections of the antigen can be administered to improve antibody production. Following immunization, antigen-specific B cells are harvested, e.g., from the spleen and/or lymphoid tissue. For generating monoclonal antibodies, the B cells are fused with myeloma cells, which are subsequently screened for antigen specificity.

The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Additionally, phage or yeast display technology can be used to identify antibodies and heteromeric Fab fragments that specifically bind to selected antigens (see. e.g., McCafferty et al., Nature 348:552-554 (1990); Marks et al., Biotechnology 10:779-783 (1992); Lou et al., PEDS 23:311 (2010); and Chao et al., Nature Protocols 1:755-768 (2006)). Alternatively, antibodies and antibody sequences may be isolated and/or identified using a yeast-based antibody presentation system, such as that disclosed in, e.g., Xu et al., Protein Eng Des Sel, 2013, 26:663-670: WO 2009/036379; WO 2010/105256; and WO 2012/009568. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can also be adapted to produce antibodies.

Antibodies can be produced using any number of expression systems, including prokaryotic and eukaryotic expression systems. In some embodiments, the expression system is a mammalian cell, such as a hybridoma, or a CHO cell. Many such systems are widely available from commercial suppliers. In embodiments in which an antibody comprises both a VH and VL region, the VH and VL regions may be expressed using a single vector, e.g., in a di-cistronic expression unit, or be under the control of different promoters. In other embodiments, the VH and VL region may be expressed using separate vectors.

In some embodiments, an anti-15-PGDH antibody comprises one or more CDR, heavy chain, and/or light chain sequences that are affinity matured. For chimeric antibodies, methods of making chimeric antibodies are known in the art. For example, chimeric antibodies can be made in which the antigen binding region (heavy chain variable region and light chain variable region) from one species, such as a mouse, is fused to the effector region (constant domain) of another species, such as a human. As another example, “class switched” chimeric antibodies can be made in which the effector region of an antibody is substituted with an effector region of a different immunoglobulin class or subclass.

In some embodiments, an anti-15-PGDH antibody comprises one or more CDR, heavy chain, and/or light chain sequences that are humanized. For humanized antibodies, methods of making humanized antibodies are known in the art. See, e.g., U.S. Pat. No. 8,095,890. Generally, a humanized antibody has one or more amino acid residues introduced into it from a source which is non-human. As an alternative to humanization, human antibodies can be generated. As a non-limiting example, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See. e.g., Jakobovits et al., Proc. Natl. Acad. Sci. USA, 90:2551 (1993); Jakobovits et al., Nature, 362:255-258 (1993); Bruggermann et al., Year in Immun., 7:33 (1993); and U.S. Pat. Nos. 5,591,669, 5,589,369, and 5,545,807.

In some embodiments, antibody fragments (such as a Fab, a Fab′, a F(ab′)2, a scFv, nanobody, or a diabody) are generated. Various techniques have been developed for the production of antibody fragments, such as proteolytic digestion of intact antibodies (see, e.g., Morimoto et al., J. Biochem. Biophys. Meth., 24:107-117 (1992); and Brennan et al., Science, 229:81 (1985)) and the use of recombinant host cells to produce the fragments. For example, antibody fragments can be isolated from antibody phage libraries. Alternatively, Fab′-SH fragments can be directly recovered from E. coli cells and chemically coupled to form F(ab′)₂ fragments (see. e.g., Carter et al., BioTechnology, 10:163-167 (1992)). According to another approach, F(ab′)2 fragments can be isolated directly from recombinant host cell culture. Other techniques for the production of antibody fragments will be apparent to those skilled in the art.

Methods for measuring binding affinity and binding kinetics are known in the art.

These methods include, but are not limited to, solid-phase binding assays (e.g., ELISA assay), immunoprecipitation, surface plasmon resonance (e.g., Biacore™ (GE Healthcare. Piscataway, NJ)), kinetic exclusion assays (e.g., KinExA®), flow cytometry, fluorescence-activated cell sorting (FACS), BioLayer interferometry (e.g., Octet™ (FortdBio, Inc., Menlo Park, CA)), and western blot analysis.

### Peptides

In some embodiments, the agent is a peptide, e.g., a peptide that binds to and/or inhibits the enzymatic activity or stability of 15-PGDH. In some embodiments, the agent is a peptide aptamer. Peptide aptamers are artificial proteins that are selected or engineered to bind to specific target molecules. Typically, the peptides include one or more peptide loops of variable sequence displayed by the protein scaffold. Peptide aptamer selection can be made using different systems, including the yeast two-hybrid system. Peptide aptamers can also be selected from combinatorial peptide libraries constructed by phage display and other surface display technologies such as mRNA display, ribosome display, bacterial display and yeast display. See, e.g., Reverdatto et al., 2015, Curr. Top. Med. Chem. 15:1082-1101.

In some embodiments, the agent is an affimer. Affimers are small, highly stable proteins, typically having a molecular weight of about 12-14 kDa, that bind their target molecules with specificity and affinity similar to that of antibodies. Generally, an affimer displays two peptide loops and an N-terminal sequence that can be randomized to bind different target proteins with high affinity and specificity in a similar manner to monoclonal antibodies. Stabilization of the two peptide loops by the protein scaffold constrains the possible conformations that the peptides can take, which increases the binding affinity and specificity compared to libraries of free peptides. Affimers and methods of making affimers are described in the art. See, e.g., Tiede et al., eLife, 2017, 6:e24903. Affimers are also commercially available, e.g., from Avacta Life Sciences.

### Vectors and Modified RNA

In some embodiments, polynucleotides providing 15-PGDH inhibiting activity, e.g., a nucleic acid inhibitor such as an siRNA or shRNA, or a polynucleotide encoding a polypeptide that inhibits 15-PGDH, are introduced into cells, e.g., tissue cells, using an appropriate vector. Examples of delivery vectors that may be used with the present disclosure are viral vectors, plasmids, exosomes, liposomes, bacterial vectors, or nanoparticles. In some embodiments, any of the herein-described 15-PGDH inhibitors, e.g., a nucleic acid inhibitor or a polynucleotide encoding a polypeptide inhibitor, are introduced into cells, e.g., tissue cells, using vectors such as viral vectors. Suitable viral vectors include but not limited to adeno-associated viruses (AAVs), adenoviruses, and lentiviruses. In some embodiments, a 15-PGDH inhibitor, e.g., a nucleic acid inhibitor or a polynucleotide encoding a polypeptide inhibitor, is provided in the form of an expression cassette, typically recombinantly produced, having a promoter operably linked to the polynucleotide sequence encoding the inhibitor. In some cases, the promoter is a universal promoter that directs gene expression in all or most tissue types; in other cases, the promoter is one that directs gene expression specifically in cells of the tissue being targeted.

In some embodiments, the nucleic acid or protein inhibitors of 15-PGDH are introduced into a subject, e.g., into the tissues of a subject, using modified RNA. Various modifications of RNA are known in the art to enhance, e.g., the translation, potency and/or stability of RNA, e.g., shRNA or mRNA encoding a 15-PGDH polypeptide inhibitor, when introduced into cells of a subject. In particular embodiments, modified mRNA (mmRNA) is used, e.g., mmRNA encoding a polypeptide inhibitor of 15-PGDH. In other embodiments, modified RNA comprising an RNA inhibitor of 15-PGDH expression is used, e.g., siRNA, shRNA, or miRNA. Non-limiting examples of RNA modifications that can be used include anti-reverse-cap analogs (ARCA), polyA tails of, e.g., 100-250 nucleotides in length, replacement of AU-rich sequences in the 3′UTR with sequences from known stable mRNAs, and the inclusion of modified nucleosides and structures such as pseudouridine, e.g., N1-methylpseudouridine, 2-thiouridine, 4′thioRNA, 5-methylcytidine, 6-methyladenosine, amide 3 linkages, thioate linkages, inosine, 2′-deoxyribonucleotides, 5-Bromo-uridine and 2′-O-methylated nucleosides. A non-limiting list of chemical modifications that can be used can be found, e.g., in the online database crdd.osdd.net/servers/simamod/. RNAs can be introduced into cells in vivo using any known method, including, inter alia, physical disturbance, the generation of RNA endocytosis by cationic carriers, electroporation, gene guns, ultrasound, nanoparticles, conjugates, or high-pressure injection. Modified RNA can also be introduced by direct injection, e.g., in citrate-buffered saline. RNA can also be delivered using self-assembled lipoplexes or polyplexes that are spontaneously generated by charge-to-charge interactions between negatively charged RNA and cationic lipids or polymers, such as lipoplexes, polyplexes, polycations and dendrimers. Polymers such as poly-L-lysine, polyamidoamine, and polyethyleneimine, chitosan, and poly(β)-amino esters) can also be used. See, e.g., Youn et al. (2015) Expert Opin Biol Ther, September 2; 15(9): 1337-1348; Kaczmarek et al. (2017) Genome Medicine 9:60; Gan et al. (2019) Nature comm. 10: 871; Chien et al. (2015) Cold Spring Harb Perspect Med. 2015; 5:a014035; the entire disclosures of each of which are herein incorporated by reference.

### 4. Methods of Administration

In one aspect, the present disclosure provides a method of improving, enhancing, and/or rejuvenating neuromuscular junction (NMJ) morphology and/or function, and/or inducing and/or promoting formation of NMJs, in a subject having degeneration of NMJs, the method comprising: administering to the subject an amount of a 15-PGDH inhibitor effective to inhibit 15-PGDH activity and/or reduce 15-PGDH levels in the subject, thereby improving, enhancing, and/or rejuvenating NMJ morphology and/or function, and/or inducing and/or promoting formation of NMJs, in the subject. In some embodiments, the method results in increased pre-synaptic motor neuron and postsynaptic AChR juxtaposition and/or connectivity. In some embodiments, the method results in a decreased number of fragmented acetylcholine receptor (AChR) clusters at the NMJ. In some embodiments, the method results in a decreased number of NMJs lacking the presence of motor neurons. In some embodiments, the method results in decreased blebbing of motor neuron axons. In some embodiments, the method results in decreased apoptosis of motor neurons. In some embodiments, the method results in enhanced NMJ morphology, and/or increased functional conduction of nerve signals to the muscle. In some embodiments, the method results in a decreased number of AChR-rich vesicles at the NMJ. In some embodiments, the method results in increased expression and/or localization of AChR at the NMJ. In some embodiments, the method results in decreased AChR degradation. In some embodiments, the method results in increased AChR stability. In some embodiments, the method results in improved, enhanced, and/or rejuvenated mitochondrial morphology in motor neuron axon terminals at the NMJ. In some embodiments, the method results in improved, enhanced, and/or rejuvenated motor neuron synaptic terminals at the NMJ. In some embodiments, the method results in improved, enhanced, and/or rejuvenated skeletal muscle mass and/or neuromuscular function in the subject.

The compounds described herein can be administered locally in the subject or systemically. In some embodiments, the compounds can be administered, for example, intraperitoneally, intramuscularly, intra-arterially, orally, intravenously, intracranially, intrathecally, intraspinally, intralesionally, intranasally, subcutaneously, intracerebroventricularly, topically, and/or by inhalation. In an example, the compounds are administered intramuscularly, e.g., by intramuscular injection. In some embodiments, the administering comprises systemic administration or local administration.

In some embodiments, the compound is administered in accordance with an acute regimen. In certain instances, the compound is administered to the subject once. In other instances, the compound is administered at one time point, and administered again at a second time point. In yet other instances, the compound is administered to the subject repeatedly (e.g., once or twice daily) as intermittent doses over a short period of time (e.g., 2 days, 3 days, 4 days, 5 days, 6 days, a week, 2 weeks, 3 weeks, 4 weeks, a month, or more). In some cases, the time between compound administrations is about 1 day, 2 days, 3 days, 4 days, 5 days, 6 days, a week, 2 weeks, 3 weeks, 4 weeks, a month, or more. In other embodiments, the compound is administered continuously or chronically in accordance with a chronic regimen over a desired period of time. For instance, the compound can be administered such that the amount or level of the compound is substantially constant over a selected time period.

In some embodiments, the 15-PGDH inhibitor is administered for at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, or at least 61 days. In some embodiments, the 15-PGDH inhibitor is administered for at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 months.

In some embodiments, the 15-PGDH inhibitor is administered for about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, or about 61 days. In some embodiments, the 15-PGDH inhibitor is administered for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, or about 25 months.

In some embodiments, the 15-PGDH inhibitor is administered for at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, at most 50, at most 51, at most 52, at most 53, at most 54, at most 55, at most 56, at most 57, at most 58, at most 59, at most 60, or at most 61 days. In some embodiments, the 15-PGDH inhibitor is administered for at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, or at most 25 months.

Administration of the compound into a subject can be accomplished by methods generally used in the art. The quantity of the compound introduced may take into consideration factors such as sex, age, weight, the types of disease or disorder, stage of the disorder, and the quantity needed to produce the desired result. Generally, for administering the compound for therapeutic purposes, the cells are given at a pharmacologically effective dose. By “pharmacologically effective amount” or “pharmacologically effective dose” is an amount sufficient to produce the desired physiological effect or amount capable of achieving the desired result, particularly for treating the condition or disease, including reducing or eliminating one or more symptoms or manifestations of the condition or disease.

The compounds described herein may be administered locally by injection into the tissue being targeted, or by administration in proximity to the tissue being targeted.

In some embodiments, the method is independent of muscle stem cell proliferation. In some embodiments, the method does not involve the inhibition of myostatin.

In some embodiments, the methods described herein involve administering to a subject having degeneration of NMJs (e.g., due to muscle denervation) an amount of a 15-PGDH inhibitor effective to inhibit 15-PGDH activity and/or reduce 15-PGDH levels in the subject. In some embodiments, the methods result in improved, enhanced, and/or rejuvenated NMJ morphology and/or function. In some embodiments, the methods result in an increase in the number of new NMJs. In some embodiments, the methods result in induced and/or promoted formation of NMJs. In some embodiments, the methods result in increased pre-synaptic motor neuron and postsynaptic AChR juxtaposition and/or connectivity (e.g., as determined by the presence of neurofilament and synaptic vesicles (markers of the pre-synaptic motor neuron axon and synapse, respectively) and by bungarotoxin-staining (BTX) which binds to AChR on the post-synaptic sarcolemma of the muscle fiber in a characteristic “pretzel” morphology). In some embodiments, the methods result in a decreased number of fragmented acetylcholine receptor (AChR) clusters at the NMJ. In some embodiments, the methods result in decreased number of NMJs lacking the presence of motor neurons. In some embodiments, the methods result in decreased blebbing of motor neuron axons. In some embodiments, the method results in decreased apoptosis of motor neurons. In some embodiments, the methods result in enhanced NMJ morphology (e.g., as determined by interconnected AChR morphology (e.g., pretzel-shaped), on the muscle fiber). In some embodiments, the methods result in a decreased number of AChR-rich vesicles at the NMJ. In some embodiments, the methods result in increased expression and/or localization of AChR at the NMJ. In some embodiments, the methods result in decreased AChR degradation. In some embodiments, the methods result in increased AChR stability. In some embodiments, the methods result in improved, enhanced, and/or rejuvenated mitochondrial morphology in motor neuron axon terminals at the NMJ. In some embodiments, the methods result in improved, enhanced, and/or rejuvenated motor neuron synaptic terminals at the NMJ. In some embodiments, the methods result in improved, enhanced, and/or rejuvenated skeletal muscle mass and/or neuromuscular function in the subject.

In some embodiments, the method described herein can be used in a combination treatment. In some embodiments, the combination treatment comprises administering an amount of a 15-PGDH inhibitor effective to inhibit 15-PGDH activity and/or reduce 15-PGDH levels in the subject in combination with an agent for treating spinal muscular atrophy (SMA), e.g., onasemnogene abeparvovec (e.g., Zolgensma®), risdiplam (e.g., Ervysdi®), or nusinersen (e.g., Spinraza®). In some embodiments, the combination treatment comprises administering an amount of a 15-PGDH inhibitor effective to inhibit 15-PGDH activity and/or reduce 15-PGDH levels in the subject in combination with an agent for treating Duchenne muscular dystrophy (DMD), e.g., prednisone, deflazacort, or eteplirsen.

### 5. Subject

In one aspect, the present disclosure provides a method of improving, enhancing, and/or rejuvenating neuromuscular junction (NMJ) morphology and/or function, and/or inducing and/or promoting formation of NMJs, in a subject having degeneration of NMJs. In some embodiments, the subject has muscle denervation, and/or partial denervation. In some embodiments, the subject has a neurogenic myopathy, an aged-induced loss of muscle mass, a genetic neuromuscular wasting disorder, nerve trauma or injury, muscle trauma or injury, or any combination thereof. In some embodiments, the genetic neuromuscular wasting disorder is spinal muscular atrophy (SMA), Duchenne muscular dystrophy (DMD), or amyotrophic lateral sclerosis (ALS).

In some embodiments, the subject has or has experienced one or more symptoms selected from the group consisting of: acute peripheral nerve injury, muscle disuse, myopathy with neurogenic and autoimmune involvement with target fibers or tubular aggregate formation, and vascular myopathy. In some embodiments, the acute peripheral nerve injury is selected from the group consisting of: contusion injury, compression-decompression injury, nerve cut, botulinum toxicity, injury due to tenotomy, and/or sports injury. In some embodiments, the compression-decompression injury is selected from the group consisting of: edema, carpal tunnel syndrome, Baker's cyst, and repetitive task injury. In some embodiments, the muscle disuse is selected from the group consisting of: immobilization after bone fracture, prolonged bed rest, recovery after surgery, recovery from ventilator use (e.g., ventilator use due to severe lung complications from pneumonia, severe COVID-19), space flight, and sedentary life-style

In some embodiments, the myopathy with neurogenic and autoimmune involvement with target fibers or tubular aggregate formation is selected from the group consisting of: Duchenne muscular dystrophy, Becker muscular dystrophy, limb girdle muscular dystrophy, central core disease, distal motor axonal neuropathy, multifocal motor neuropathy, amyotrophic lateral sclerosis, spinal muscular atrophy, multiple sclerosis, ataxia, myotonic dystrophy, neurogenic amyloidosis, proximal myopathy with tubular aggregates, rheumatoid arthritis, Sjögren's syndrome, and myasthenia gravis.

In some embodiments, the myasthenia gravis is selected from the group consisting of: congenital myasthenia gravis, episodic myasthenia gravis, and Lambert-Eaton myasthenic syndrome.

In some embodiments, the subject is a human subject. In various embodiments, the human subject is a male human. In some embodiments, the human subject is a female human. In some aspects, the human subject identifies as a man, woman, or nonbinary. In some embodiments, the human subject is less than 60 years old. In some embodiments, the human subject is less than 50 years old. In some embodiments, the human subject is less than 40 years old. In some embodiments, the human subject is less than 30 years old. In various embodiments, the human subject is about 17 years old to about 60 years old. In some embodiments, the human subject is about 17 years old to about 50 years old. In some embodiments, the human subject is about 17 years old to about 40 years old. In some embodiments, the human subject is about 17 years old to about 30 years old. In various embodiments, the human subject is about 18 years old to about 60 years old. In some embodiments, the human subject is about 18 years old to about 50 years old. In some embodiments, the human subject is about 18 years old to about 40 years old. In some embodiments, the human subject is about 18 years old to about 30 years old. In various embodiments, the human subject is at least 15 years old. In some embodiments, the human subject is at least 17 years old. In some embodiments, the human subject is at least 18 years old. In some embodiments, the human subject is at least 20 years old. In some embodiments, the human subject is at least 25 years old.

In some embodiments, the subject is less than 10, less than 11, less than 12, less than 13, less than 14, less than 15, less than 16, less than 17, less than 18, less than 19, less than 20, less than 21, less than 22, less than 23, less than 24, less than 25, less than 26, less than 27, less than 28, less than 29, less than 30, less than 31, less than 32, less than 33, less than 34, less than 35, less than 36, less than 37, less than 38, less than 39, less than 40, less than 41, less than 42, less than 43, less than 44, less than 45, less than 46, less than 47, less than 48, less than 49, less than 50, less than 51, less than 52, less than 53, less than 54, less than 55, less than 56, less than 57, less than 58, less than 59, or less than 60 years of age. In some embodiments, the subject is less than 30 years of age.

In some embodiments, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, or at least 60 years of age. In some embodiments, the subject is at least 30 years of age.

### 6. Pharmaceutical Compositions

The pharmaceutical compositions of the compounds described herein may comprise a pharmaceutically acceptable carrier. In certain aspects, pharmaceutically acceptable carriers are determined in part by the particular composition being administered, as well as by the particular method used to administer the composition. Accordingly, there is a wide variety of suitable formulations of pharmaceutical compositions described herein (see, e.g., REMINGTON'S PHARMACEUTICAL SCIENCES, 18TH ED., Mack Publishing Co., Easton, PA (1990)).

As used herein, “pharmaceutically acceptable carrier” comprises any of standard pharmaceutically accepted carriers known to those of ordinary skill in the art in formulating pharmaceutical compositions. Thus, the compounds, by themselves, such as being present as pharmaceutically acceptable salts, or as conjugates, may be prepared as formulations in pharmaceutically acceptable diluents; for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or the like, or as solid formulations in appropriate excipients.

The pharmaceutical compositions will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants (e.g., ascorbic acid, sodium metabisulfite, butylated hydroxytoluene, butylated hydroxyanisole, etc.), bacteriostats, chelating agents such as EDTA or glutathione, solutes that render the formulation isotonic, hypotonic or weakly hypertonic with the blood of a recipient, suspending agents, thickening agents, preservatives, flavoring agents, sweetening agents, and coloring compounds as appropriate.

The pharmaceutical compositions described herein are administered in a manner compatible with the dosage formulation, and in such amount as will be therapeutically effective. The quantity to be administered depends on a variety of factors including, e.g., the age, body weight, physical activity, and diet of the individual, the condition or disease to be treated, and the stage or severity of the condition or disease. In certain embodiments, the size of the dose may also be determined by the existence, nature, and extent of any adverse side effects that accompany the administration of a therapeutic agent(s) in a particular individual.

It should be understood, however, that the specific dose level and frequency of dosage for any particular patient may be varied and may depend upon a variety of factors including the activity of the specific compound employed, the metabolic stability and length of action of that compound, the age, body weight, hereditary characteristics, general health, sex, diet, mode and time of administration, rate of excretion, drug combination, the severity of the particular condition, and the host undergoing therapy.

In certain embodiments, the dose of the compound may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, preferably in unit dosage forms suitable for simple administration of precise dosages.

As used herein, the term “unit dosage form” refers to physically discrete units suitable as unitary dosages for humans and other mammals, each unit containing a predetermined quantity of a therapeutic agent calculated to produce the desired onset, tolerability, and/or therapeutic effects, in association with a suitable pharmaceutical excipient (e.g., an ampoule). In addition, more concentrated dosage forms may be prepared, from which the more dilute unit dosage forms may then be produced. The more concentrated dosage forms thus will contain substantially more than, e.g., at least 1, 2, 3, 4, 5, 6, 7, 8, 9, 10, or more times the amount of the therapeutic compound.

Methods for preparing such dosage forms are known to those skilled in the art (see. e.g., REMINGTON'S PHARMACEUTICAL SCIENCES, supra). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., REMINGTON'S PHARMACEUTICAL SCIENCES, supra).

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The dosage forms can additionally include lubricating agents such as talc, magnesium stearate, and mineral oil; wetting agents; emulsifying agents; suspending agents; preserving agents such as methyl-, ethyl-, and propyl-hydroxy-benzoates (e.g., the parabens); pH adjusting agents such as inorganic and organic acids and bases; sweetening agents, and flavoring agents. The dosage forms may also comprise biodegradable polymer beads, dextran, and cyclodextrin inclusion complexes.

For oral administration, the therapeutically effective dose can be in the form of tablets, capsules, emulsions, suspensions, solutions, syrups, sprays, lozenges, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

The therapeutically effective dose can also be provided in a lyophilized form. Such dosage forms may include a buffer, e.g., bicarbonate, for reconstitution prior to administration, or the buffer may be included in the lyophilized dosage form for reconstitution with, e.g., water. The lyophilized dosage form may further comprise a suitable vasoconstrictor, e.g., epinephrine. The lyophilized dosage form can be provided in a syringe, optionally packaged in combination with the buffer for reconstitution, such that the reconstituted dosage form can be immediately administered to an individual.

In some embodiments, additional compounds or medications can be co-administered to the subject. Such compounds or medications can be co-administered for the purpose of alleviating signs or symptoms of the disease being treated, reducing side-effects caused by induction of the immune response, etc.

### 7. Kits

Other embodiments of the compositions described herein are kits comprising a 15-PGDH inhibitor. The kit typically contains containers, which may be formed from a variety of materials such as glass or plastic, and can include for example, bottles, vials, syringes, and test tubes. A label typically accompanies the kit, and includes any writing or recorded material, which may be electronic or computer readable form providing instructions or other information for use of the kit contents.

In some embodiments, the kit comprises one or more reagents for improving, enhancing, and/or rejuvenating neuromuscular junction morphology and/or function, and/or inducing and/or promoting formation of NMJs in a subject having degeneration of NMJs. In some embodiments, the kit comprises one or more reagents for the treatment of a neurogenic myopathy, an aged-induced loss of muscle mass, a genetic neuromuscular wasting disorder, and/or muscle trauma or injury. In some embodiments, the kit comprises an agent that antagonizes the expression or activity of 15-PGDH. In some embodiments, the kit comprises an inhibitory nucleic acid (e.g., an antisense RNA, small interfering RNA (siRNA), microRNA (miRNA), short hairpin RNA (shRNA)), or a polynucleotide encoding a 15-PGDH inhibiting polypeptide, that inhibits or suppresses 15-PGDH mRNA or protein expression or activity, e.g., enzyme activity. In some embodiments, the kit comprises a modified RNA, e.g., a modified shRNA or siRNA, or a modified mRNA encoding a polypeptide 15-PGDH inhibitor. In some embodiments, the kit further comprises one or more plasmid, bacterial or viral vectors for expression of the inhibitory nucleic acid or polynucleotide encoding a 15-PGDH-inhibiting polypeptide. In some embodiments, the kit comprises an antisense oligonucleotide capable of hybridizing to a portion of a 15-PGDH-encoding mRNA. In some embodiments, the kit comprises an antibody (e.g., a monoclonal, polyclonal, humanized, bispecific, chimeric, blocking or neutralizing antibody) or antibody-binding fragment thereof that specifically binds to and inhibits a 15-PGDH protein. In some embodiments, the kit comprises a blocking peptide. In some embodiments, the kit comprises an aptamer (e.g., a peptide or nucleic acid aptamer). In some embodiments, the kit comprises an affimer. In some embodiments, the kit comprises a modified RNA. In particular embodiments, the kit comprises a small molecule inhibitor. e.g., SW033291, that binds to 15-PGDH or inhibits its enzymatic activity. In some embodiments, the kit further comprises one or more additional therapeutic agents, e.g., agents for administering in combination therapy with the agent that antagonizes the expression or activity of 15-PGDH.

In some embodiments, the kits can further comprise instructional materials containing directions (e.g., protocols) for the practice of the methods described herein (e.g., instructions for using the kit for improving, enhancing, and/or rejuvenating neuromuscular junction morphology and/or function, and/or inducing and/or promoting formation of NMJs, in a subject having degeneration of NMJs; and/or for using the kit for the treatment of a neurogenic myopathy, an aged-induced loss of muscle mass, a genetic neuromuscular wasting disorder, and/or muscle trauma or injury). While the instructional materials typically comprise written or printed materials, they are not limited to such. Any medium capable of storing such instructions and communicating them to an end user is contemplated by this disclosure. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The present disclosure will be described in greater detail byway of specific examples. The following examples are offered for illustrative purposes only, and are not intended to limit the disclosure in any manner. Those of skill in the art will readily recognize a variety of noncritical parameters which can be changed or modified to yield essentially the same results.

### Example 1. Targeting the Prostaglandin E2 Degrading Enzyme, 15-PGDH, to Promote Muscle Function in Spinal Muscular Atrophy

Although progressive motor neuron loss in spinal muscular atrophy (SMA) is now controllable by three distinct treatments, most patients have persistent motor deficits that necessitate supplementary measures. Halting disease progression is not enough. SMA patients' muscles remain underdeveloped, and patients require therapeutic interventions that augment their muscle mass and strength to enhance their mobility. The present disclosure provides methods to improve, treat, or alleviate symptoms in aging-related muscle wasting which can be applied to SMA. As described herein, 15-PGDH inhibition leads to remodeling of neuromuscular junctions in mice. As described herein, inhibition of 15-PGDH can provide a method or treatment to improve, enhance, and/or rejuvenate NMJ following denervation. Systemic 15-PGDH inhibition can increase muscle mass and strength in by improving, enhancing, and/or rejuvenating mitochondrial function in muscle and in motor neurons. Further, the method described herein has the potential to improve quality of life for patients afflicted with SMA.

Spinal muscular atrophy (SMA) treatments have advanced considerably through the application of gene therapy and splicing modifiers. Treatment with antisense oligonucleotides (Spinraza), splicing modifiers (Risdiplam) and AAV9-SMN1 gene therapy (Zolgensma) have greatly improved patient quality of life through promoting the survival of motor neurons, however, patients are still afflicted with delays in motor milestones and function. Therapeutic strategies that augment muscle mass and strength are needed to improve SMA patient strength, mobility, and quality of life. As described herein, inhibition of 15-PGDH in aging-related muscle wasting and muscular dystrophy can also be applied to disease such as SMA. Like SMA, muscle dysfunction in aging is attributable, in part, to changes at the neuromuscular junction. Thus, therapeutics that benefit aged and sarcopenic muscle wasting can also benefit SMA patients after gene therapy.

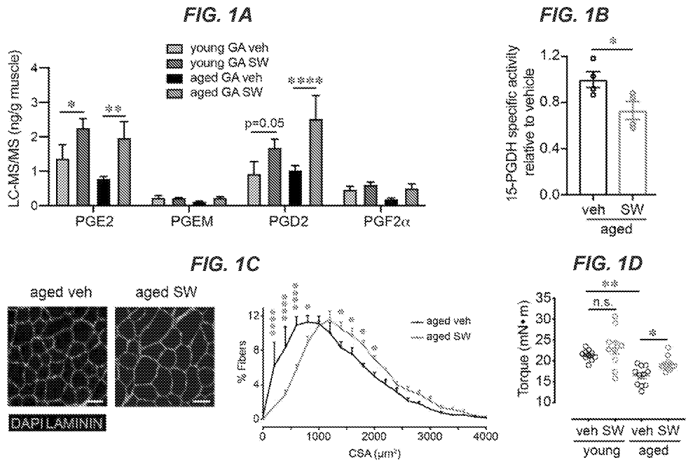

The prostaglandin E2 catabolizing enzyme, 15-hydroxyprostaglandin dehydrogenase (15-PGDH), is a pivotal regulator of muscle aging and that its inhibition leads to a marked increase in muscle mass and strength in aged mice¹. 15-PGDH is a nicotinamide adenine dinucleotide (NAD+) dependent enzyme that increases with aging in muscle and catabolizes PGE2. FIGS. 1A-1B show increased 15-PGDH activity along with PGE2 levels, and these are reduced in aged mouse muscle. As shown in FIGS. 1C-1D, systemic inhibition of 15-PGDH using the small molecule SW033291 (SW) for one month improves, enhances, and/or rejuvenates muscle PGE2 levels to the physiological levels measured in young mice in parallel with a 15-20% increase in muscle mass and force. Strikingly, overexpression of 15-PGDH in young mouse muscles leads to loss of muscle mass and strength after just one month¹. This underscores the importance of 15-PGDH as a previously unrecognized “master regulator of muscle wasting”.

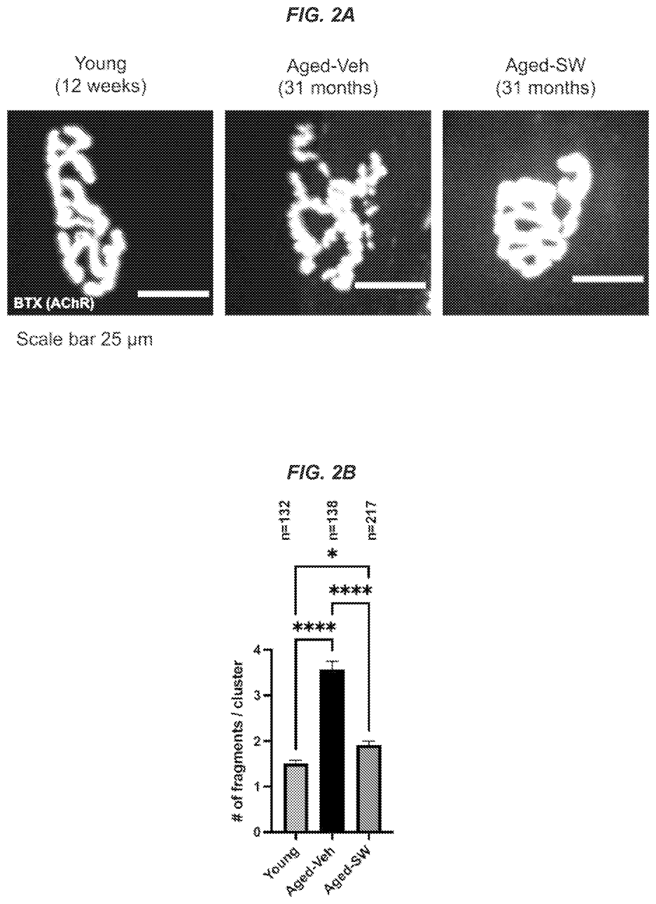

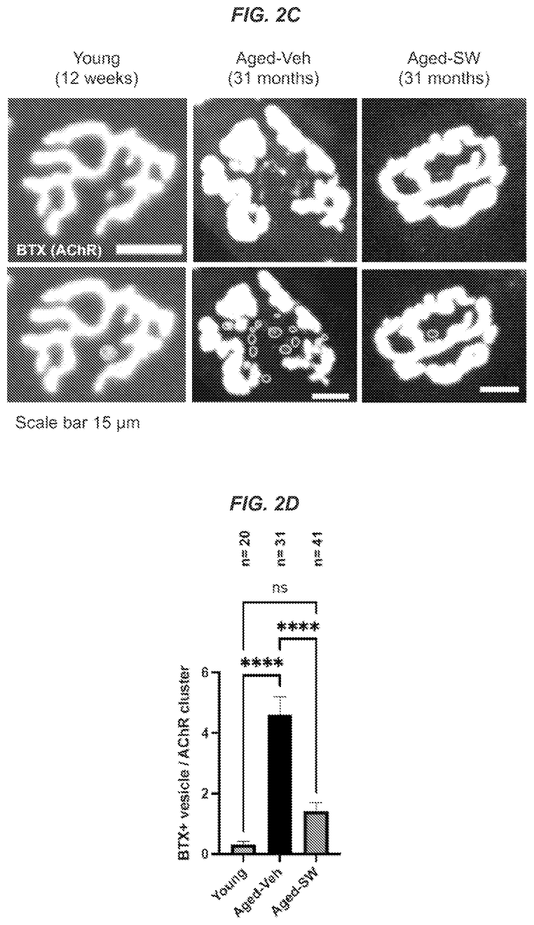

As denervation is a hallmark of aging muscle, the effect of systemic 15-PGDH inhibition on aged neuro-muscular junctions (NMJs) was investigated. As shown in FIGS. 2A-2D, one month of 15-PGDH inhibition improves, enhances, and/or rejuvenates the morphology of aged neuro-muscular junctions (NMJs) in mice. FIG. 2A shows that aged NMJs exhibit pathological structural changes which includes acetylcholine receptor (AChR) fragmentation, and FIG. 2B shows increased rate of receptor degradation demonstrated by increased numbers of AChR rich endo/lysosomal vesicles². These results show that one month of intraperitoneal (i.p.) administration of SW improves, enhances, and/or rejuvenates the intact morphology of the AChRs and reduces the number of AChR rich endo/lysosomal vesicles in aged mice.

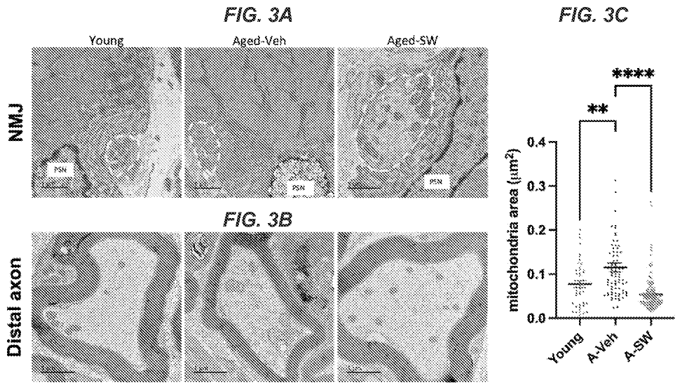

In addition, FIG. 3A shows electron microscopy analysis of aged NMJs demonstrate rejuvenated morphology of the mitochondria in motor neuron (MN) axon terminals. This results highlight that 1) NMJs are mitochondria rich to accommodate their high bioenergetic demands essential for neurotransmitter release and muscle contraction and, 2) mitochondria of lower MNs in the type III SMA mouse model exhibit distended and enlarged morphology³. FIG. 3B shows ultra-structural analysis of distal axons that innervate muscle fibers show improvement, enhancement, and/or rejuvenation of mitochondrial morphology in MNs of aged mice post SW treatments.

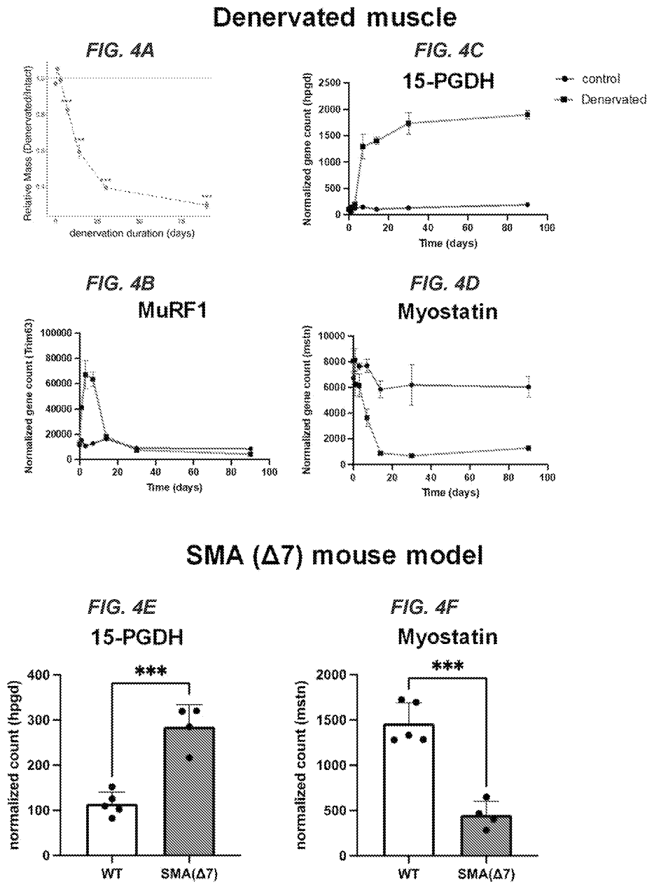

Furthermore, reanalysis of recent published studies on a long-term muscle denervation model⁴ and a severe mouse model of SMA⁵ highlights the role of 15-PGDH in denervated and SMA muscle, respectively. FIG. 4C shows that 15-PGDH gene expression increases as early as 7 days post denervation in gastrocnemius (GA) muscle, and its levels are sustained in the denervated muscle 90 days post denervation. FIG. 4C shows that increase in muscular gene expression of 15-PGDH is concurrent with decreased muscle mass as depicted in FIG. 4A. Genes that are classically expressed in muscle atrophy (atrogins and myostatin) are only increased transiently and return to pre-injury levels (MurF1 (as depicted in FIG. 4B) and Atrogin-1) or are reduced below homeostatic levels (myostatin) after 30 days of denervation (as depicted in FIG. 4D). Similar results are observed for muscle levels of 15-PGDH and myostatin in the delta7 mouse model of SMA at post-natal day (PND) 10. As shown in FIG. 4E, while muscle (tibialis anterior muscle) levels of 15-PGDH show a significant increase, FIG. 4F shows that myostatin levels were decrease at PND10 in the severe delta7 SMA mouse model. Together, these data emphasize that muscle levels of 15-PGDH are correlated with muscle denervation and are inversely correlated with muscle health and fitness in mice.

This example describes a novel therapeutic approach using 15-PGDH inhibition to enhance muscle mass and function in an SMA mouse model. A systemic decrease in 15-PGDH activity for a period of one month suffices to improve, enhance, and/or rejuvenate muscle mass, strength, and NMJ morphology and mitochondrial biogenesis in a mild type III mouse model of SMA (Smn1C/C).

As shown herein, 15-PGDH inhibition improves muscle function in several muscle atrophy conditions including aging. Ultimately, the methods described herein can positively impact quality of life, especially in existing SMA patients where gene therapy and splicing modifier treatments limit disease progression but do not improve, enhance, and/or rejuvenate atrophied muscle.

### REFERENCES

1. Palla A R, Ravichandran M, Wang Y X, Alexandrova L, Yang A V, Kraft P, Holbrook C A, Schurch C M, Ho ATV, Blau H M. Inhibition of prostaglandin-degrading enzyme 15-PGDH rejuvenates aged muscle mass and strength. Science [Internet]. American Association for the Advancement of Science; 2021 Jan. 29 [cited 2021 February 26]:371(6528). Available from: https://science.sciencemag.org/content/371/6528/eabc8059 PMID: 33303683
2. Khan M M, Strack S. Wild F. Hanashima A. Gasch A. Brohm K, Reischl M. Camio S. Labeit D, Sandri M, Labeit S, Rudolf R. Role of autophagy, SQSTM1, SH3GLB1, and TRIM63 in the turnover of nicotinic acetylcholine receptors. Autophagy. Taylor & Francis; 2014 Jan. 1:10(1):123-136. PMID: 24220501
3. Fulceri F, Biagioni F, Limanaqi F, Busceti C L, Ryskalin L, Lenzi P. Fomai F. Ultrastructural characterization of peripheral denervation in a mouse model of Type III spinal muscular atrophy. J Neural Transm. 2021 Jun. 1; 128(6):771-791.
4. Ehmsen J T, Kawaguchi R, Mi R. Coppola G, Hoke A. Longitudinal RNA-Seq analysis of acute and chronic neurogenic skeletal muscle atrophy. Sci Data. Nature Publishing Group; 2019 Sep. 24; 6(1):179.
5. McCormack N M, Villalón E, Viollet C, Soltis A R, Dalgard C L, Lorson C L, Burnett B G. Survival motor neuron deficiency slows myoblast fusion through reduced myomaker and myomixer expression. J Cachexia Sarcopenia Muscle. 2021 August; 12(4):1098-1116. PMCID: PMC8350220

### Example 2. 15-PGDH is a Hallmark of Muscle Denervation, and its Inhibition Promotes NMJ Formation

Sarcopenia is the loss of muscle mass and mobility that occurs with aging and is correlated with increased mortality. Although the molecular drivers of age-associated muscle wasting are not well understood, partial denervation of myofibers is a hallmark associated with muscle aging. As shown in this example, 15-PGDH is upregulated as a part of a sustained genetic program associated with autophagy and mitophagy in denervated myofibers. Strikingly, 15-PGDH is spatially localized in subcellular compartments that lack mitochondria in aged denervated myofibers and human neurogenic myopathies. As described herein, 15-PGDH hinders repair after denervation, as pharmacological inhibition accelerates motor recovery after sciatic nerve crush injury and improves, enhances, and/or rejuvenates neuromuscular junction health in geriatric mice. 15-PGDH can be used as a molecular target to promote muscle innervation after acute peripheral nerve injury and aging, with potential therapeutic relevance for human chronic neurogenic myopathies.

Prostaglandin E2 (PGE2) is a key regulator of muscle stem cell proliferation and an essential factor for successful muscle regeneration post-injury (1). More recently 15-PGDH, a PGE2 catabolizing enzyme, is identified as an aging factor that contributes to muscle wasting. Notably, inhibition of 15-PGDH improves, enhances, and/or rejuvenates muscle mass and function by inducing muscle mitochondrial biogenesis (2). Pharmacological inhibition of 15-PGDH with a small molecule inhibitor (SW033291; SW) leads to a reduction in catabolic regulators of muscle atrophy such as atrogenes, ubiquitin ligases MuRF1 (Trim63) and Fbxo32 (Alrogene-1), and TGF-beta signaling. However, signaling pathways that regulate 15-PGDH expression or its proteomic interactions in aged muscle fibers are not well understood.

As described herein, nerve-dependent activity plays a pivotal role in regulating 15-PGDH expression in myofibers. 15-PGDH is significantly upregulated in the myofibers upon sciatic nerve transection, which is coupled with increased PGE2 metabolism revealed by mass spectrometry. 15-PGDH is apart of a sustained genetic program that is activated in denervated myofibers, having a distinct temporal dynamic from previously characterized catabolic regulators of muscle atrophy. As shown herein, single nuclei analysis of denervated muscle revealed that 15-PGDH expression is positively correlated with autophagy and mitophagy. Intriguingly, this relationship is reflected in the subcellular compartmentalization of 15-PGDH in denervated aged muscles, in aggregates with autophagy markers LC3A that also lack mitochondria. Similar 15-PGDH aggregates are observed in target fibers of patients biopsies with neurogenic myopathies.

As described herein, in one aspect, pharmacological inhibition of 15-PGDH accelerates motor recovery and promotes formation of new NMJs after sciatic nerve crush, a mouse model of peripheral nerve injury. In geriatric mice, 1 month of 15-PGDH inhibition reverses age-related NMJ loss and improves, enhances, and/or rejuvenates the stability of AChRs evidenced by reduction of AChR degradation and fragmentation in aged NMJs.

As described herein, 15-PGDH is a molecular target of denervation that negatively regulates NMJ stability. In one aspect, pharmacological inhibition of 15-PGDH accelerates functional recovery in a mouse model of peripheral nerve injury, fosters formation of new NMJs in geriatric mice, and ameliorates abnormalities in motor neuron synapses at the NMJ. In another aspect, the present disclosure provides a novel and beneficial role for 15-PGDH inhibition in aging with therapeutic potentials in treating neurogenic muscular atrophies.

### Materials and Methods

### Animal Husbandry

All animal experiments and protocols were in compliance with the institutional guidelines of Stanford University and Administrative Panel on Laboratory Animal Care (ALPAC). Male C57BL/6 mice were used in this study. Aged mice (24-29 months old) were obtained from the US National Institute on Aging (NIA) aged colony and young mice (2-4 months old) were purchased from Jackson Laboratory.

### In Vivo Drug Treatment

### Aged Mice

Mice were treated for 1 month once a day intraperitoneally with 5 mg/kg of SW033291 (SW) or vehicle (10% ethanol, 5% Cremophor EL, and 85% D5W (5% (wt/v) dextrose in water, Tables 3 and 4)) as previously described (2). Vehicle and SW treatments were performed in seven independent experiments for aging studies that included young and aged vehicle- and SW-treated groups. Mice were randomized based on their body weight into vehicle-treated control and SW-treated experimental groups prior to the start of injections. A full-body necropsy was performed on all mice at the experimental endpoint and mice that developed tumors were excluded from the study.

### Drug Treatment in Mice Undergoing Sciatic Nerve Transection/Crush Injury

Nerve injury was induced as described below. Mice were treated with SW or vehicle as control intraperitoneally once daily for 14 days post-injury as described above.

### Sciatic Nerve Transection Surgery

Mice were injected with 1 mg/kg of body weight with Buprenorphine SR™ LAB subcutaneously 30 minutes prior to the surgery for post-op pain management. Mice were anesthetized with 3% isoflurane. The right leg was shaved from knee to hip and sterilized. A 0.5 cm incision was introduced parallel to the femur approximately 1.5 mm anterior to the femur. Hamstring muscles were located and separated using autoclaved sharp sticks to allow access to the sciatic nerve. The sciatic nerve was firmly held using hemostatic forceps (Fine Science Tools, 13020-12) and then ˜5 mm of the nerve was transected using fine sterile scissors. The incision was then closed using 7 mm autoclaved reflex clips (World Precision Instruments, 500344; Applier: 500345). Mice were monitored for recovery from anesthesia and then returned to their home cages.

### Sciatic Nerve Crush Surgery

The sciatic nerve crush surgery was adopted from the protocol described previously by Bauder and Ferguson (41). The crush surgery was performed as described above with the following modifications. After locating the sciatic nerve, the nerve was mobilized gently with sharp sticks for easy access with hemostatic forceps (Fine Science Tools, 13020-12). Next, the nerve was gently placed on the bottom jaw of the forceps and was crushed for 15 seconds at 3 clicks of the forceps (first location of the locking handle). The incision was closed using 7 mm autoclaved reflex clips (World Precision Instruments, 500344; Applier: 500345). Mice were monitored for recovery from anesthesia and then returned to their home cages.

### Whole-Mount Staining of Neuromuscular Junctions

Extensor digitorum longus (EDL) and soleus muscles were dissected and fixed in 4% PFA in PBS for 30 minutes at room temperature. Muscles were washed in PBS extensively and stored in PBS with 0.01% sodium azide (Sigma. S2002) at 4 C until processed. For whole-mount staining, EDLs were separated using the distal tendon into 4 pieces to make thinner sheets of muscles for staining. The proximal and distal tendons of either soleus or EDL pieces were removed to allow for teasing thin bundles of muscle fibers using fine forceps under a stereomicroscope. Care was given in handling tissues to avoid touching the end plate band with forceps tips. Next, myofiber bundles were incubated in PBS-T (0.3% Triton X-100 in PBS) for 1 hour and were blocked in blocking solution (5% goat serum and 0.3% Triton X-100 in PBS) supplemented with 1:50 dilution of goat anti-mouse IgG (Jackson ImmunoResearch, 115-007-003) and IgM (Jackson ImmunoResearch, 115-007-020) for 1 hour at room temperature. Tissues were then incubated with a primary antibody mix against neurofilament and synaptic vesicle (2H3 and SV, DSHB) at 5 μg/ml for a minimum of 24 hours at 4 C with gentle shaking. Tissues were then rinsed with PBS-T and incubated with a secondary antibody mix (goat anti-mouse IgG1-Cy™3, Bungarotoxin-AF™647, and Hoechst 33342) diluted in blocking solution overnight at 4° C. with gentle shaking. Tissues were washed extensively with PBS and mounted in Fluoromount-G™ mounting medium (Southern Biotech, 0100-01) and left overnight to equilibrate prior to confocal microscopy.

### Immunofluorescence Staining and Imaging of Tissue Sections

Gastrocnemius (GA) and tibialis anterior (TA) muscles were collected for immunohistological analysis of muscle cross sections. Muscles were embedded in O.C.T. after dissection and were frozen in liquid-nitrogen cooled isopentane. Frozen tissues were sectioned transversely at 10 μm and were kept in −20° C. For immunohistological analyses, frozen sections were equilibrated to room temperature, rehydrated in PBS and fixed with 4% PFA for 10 minutes at room temperature. Fixed tissues were blocked in a goat serum-based blocking solution (Reagents and Materials) supplemented with 1:50 dilution of goat anti-mouse IgG (Jackson ImmunoResearch, 115-007-003) and IgM (Jackson ImmunoResearch, 115-007-020) Fab fragments for 1 hour at room temperature before incubation with primary antibody (Table 1) overnight at 4 C. Primary antibodies were introduced at either 2-5 μg/ml in blocking solution or at a dilution recommended by the manufacturer. Sections were rinsed thoroughly with PBS-T (Reagents and Materials) and were incubated with a compatible secondary antibody for 1 hour at room temperature (Table 2). Tissues were rinsed thoroughly with PBS and nuclei were counter-stained with Hoechst 33342. Sectioned were then treated with TrueBlack® Lipofuscin following manufacturers protocol and coverslipped with Fluoromount-G™ mounting medium (Southern Biotech, 0100-01). Images were acquired using a KEYENCE BZ-X700 all-in-one fluorescence microscope with a 20×/0.75 NA or an oil immersion 63×/1.4 NA objective as described previously (2).

### Confocal Microscopy

Confocal images were acquired on a Marianas spinning disk confocal (SDC) microscopy with Intelligent Imaging Innovations software using a 20×/NA 0.75 or an oil immersion 40×/NA 0.9 objective to capture multiple focal planes at a step size of 1 μm. Images acquired using the 20× objective were analyzed to score denervation, axonal swelling, and fragmentation of AChRs. Images acquired with the oil immersion 40× objective were analyzed to quantity NMJ-associated endolysosomal vesicles stained with α-bungarotoxin-AF647. Maximum intensity projection images were created from confocal slices using NIH ImageJ software. Data acquisition and NMJ scoring were performed by two researchers blinded to treatment conditions.

### CODEX Imaging

CODEX Multiplexed Imaging was performed as described in Wang et al., 2022 Biorxiv. Fluidics exchange was performed by a CODEX PhenoCycler System (Akoya Biosciences) and imaged on a Keyence BX710 Automated Microscope (Keyence). Images were captured using a 20× Nikon 0.75NA PlanApo lens in 3D with 0.8 um Z-resolution. Images were processed by CRISP-CODEX Image Processor (github.com/will-yx/CRISP-CODEX-Processor) using blind deconvolution initialized with a Gibson-Lanni PSF for 100 iterations.

### In Vivo Muscle Force Measurement

Plantar flexion peak isometric torque (N·mm) was measured in mice as described previously (2, 42, 43). Briefly, mice were anesthetized with 3% isoflurane mixed with oxygen and legs were shaved from ankle to hip to allow for reproducible access to the tibial nerve. The foot was taped to a footplate attached to a servomotor (Aurora Scientific, 300C-LR) and the knee joint was secured to a fixed steel post. Contractions were elicited by percutaneous electrical stimulation of the tibial nerve by inserting two Pt—Ir electrode needles (Aurora Scientific) posterior to the knee joint. The peak isometric torque was achieved by injecting 0.4 mA current to the tibial nerve at a frequency of 150 Hz and 0.1 ms square wave pulse. Three tetanic measurements were performed on each muscle, with 1 minute recovery between each measurement, and chose the highest value. Force measurement acquisition was blinded and the researcher performing the force measurements was unaware of treatment conditions. Data were analyzed using Aurora Scientific Dynamic Muscle Analysis Software Suite. Force was measured longitudinally in mice undergoing unilateral sciatic nerve crush injury on days 3, 7, and 14 post-injury. Peak tetanic force generated by contralateral uninjured legs was measured in all experimental mice at all time points.

### Measurement of Prostaglandins by LC-MS/MS

Prostaglandins, PGE2, PGD2, and PGF2a, and PGE2 metabolites, 15-keto PGE2 and 13,14-dihydro-15-keto PGE2 (PGEM), were measured as described previously (2). Briefly, tissues were snap frozen in liquid nitrogen upon dissection and were kept for no more than 2 weeks in −80° C. freezer before processing for prostaglandin quantification using LC-MS/MS. Tissues were homogenized in an acetone-based homogenization buffer (acetone/water 1:1 v/v) with butylated hydroxytoluene (0.005%) to prevent oxidation. Tissues were homogenized in Lysing Matrix D tubes with 1.4 mm ceramic beads (MP Biomedicals) and a FastPrep24 homogenizer. Calibration curve preparation, the extraction procedure, LC-MS/MS, and quantitative data analysis were performed exactly as described in 7.

### Western Protein Analysis

Tissues were snap-frozen upon dissection in liquid nitrogen and were stored in −80° C. freezer prior to homogenization. For total lysate preparation, tissues were homogenized in RIPA buffer (Cell Signaling Technology, 9806) with Halt™ Protease and Phosphatase Inhibitor cocktail (ThermoFisher, 78440) using Lysing Matrix S (MP Biomedicals) metal beads in a FastPrep24 homogenizer. Lysates were analyzed for total protein concentration using the BCA protein assay kit (ThermoFisher). 20 μg of total protein was analyzed on NuPAGE™ mini protein gels. Proteins were transferred to nitrocellulose membrane and stained with Ponceau S (Sigma) staining solution prior to blocking. Membranes were blocked with bovine serum albumin (BSA) based blocking solution. The following primary antibodies were used: 15-PGDH (Santa Cruz Biotechnology, sc-271418), GAPDH (Cell Signaling Technology, 2118), and β-tubulin (Cell Signaling Technology, 2146). We used the following horseradish peroxidase (HRP) conjugated secondary antibodies: anti-mouse IgG γ chain antibody (Sigma, AP503P) and anti-rabbit IgG antibody (Cell Signaling Technology, 7074). Chemiluminescence was performed using ECL substrate (ThermoFisher) with a ChemiDoc (BioRad) imaging system. Images were analyzed using NIH ImageJ software.

### Activity Assay

Gastrocnemius muscles were snap frozen upon dissection in liquid nitrogen and stored in −80° C. 15-PGDH activity was analyzed using the PicoProbe 15-PGDH Activity Assay Kit (BioVision, K562) according to the manufacturer's manual. Briefly, tissues were homogenized in Lysing Matrix D tubes with 1.4 mm ceramic beads (MP Biomedicals) and FastPrep homogenizer the assay buffer provided with the kit. Tissue homogenates were spun at 10*000 g for 5 minutes at 4 C and supernatant was collected and used for 15-PGDH activity measurement. Supernatants were also analyzed for total protein concentration using BCA protein assay kit (ThermoFisher) and the measured 15-PGDH activity was normalized to total protein amounts.

### Single Nuclei RNA-Seq

Muscle nuclei were isolated using a modified version of 10× Genomics “Nuclei Isolation from Complex Tissues for Single Cell Multiome ATAC+Gene Expression Sequencing” protocol. In brief, −50 mg of snap frozen muscles were minced in 200 μL of ice cold NP-40 Lysis Buffer until homogenous. Another 300 μL of NP-40 Lysis buffer was added, dounce homogenized in a 1.5 mL Eppendorf 10 times, and lysed over 5 mins on ice. The suspension was filtered through a 40 μm filter cap FACS tube, washed with 250 μL of nuclei FACS buffer (1% BSA+0.2 U/μL RNAse inhibitor in PBS) then labelled with 7AAD (Miltenyi) for 2 mins. Nuclei were pelleted at 300 g for 10 mins at 4° C. using a swinging bucket centrifuge, then resuspended in 250 μL of nuclei FACS buffer. 7AAD+ nuclei were prospectively isolated by FACS (Sony SH800 equipped with 4 lasers). Nuclei were permeabilized in 0.1× Lysis Buffer, washed with 1 mL of wash buffer, pelleted and resuspended in nuclei buffer to 6000 nuclei per μL before droplet generation using a 10× Chromium System (10× Genomics). Library generation was performed per manufacturer's instructions and sequenced on an Illumina NovaSeq 6000.

### Reanalysis of Denervated Muscle Bulk RNA-Seq Data

Longitudinal RNA-seq analysis of skeletal muscle denervation after SNT was performed on data deposited by Ehmsen et al., 2019 Scientific Data from SRP196460. Sequence alignment to mm10 was performed using HiSat2 and mRNA counts were generated by StringTie. DESeq2 was used to calculate transcripts per million and differentially expressed genes (DEGs). 55 of 56 samples passed quality control after PCA analysis. Time series gene set enrichment analysis was performed on all DEGs by hierarchical clustering normalized temporal expression across time points.

### Spinal Cord Immunostaining

Spinal cords were harvested as previously described using hydraulic extrusion of the entire intact spinal cord. Briefly, mice were euthanized with carbon dioxide and decapitated using scissors caudally to the brain stem. The spinal columns were severed just caudal to the sacral spinal cord. A blunt 25-gauge syringe containing ice-cold PBS was inserted into the caudal end of the spinal cord to hydraulicly extrude the entire cord in a petri0dish with ice-cold PBS. The lumbar region was identified under a stereomicroscope, dissected with a sharp blade, and fixed overnight at 4 C in 4% PFA in phosphate buffer. Fixed lumbar spinal cords were embedded in O.C.T. and were frozen in liquid-nitrogen-cooled isopentane. Frozen tissues were sectioned transversely at 35 μm and were kept in −20 C. For immunohistological analyses, frozen sections were equilibrated to room temperature, rehydrated in PBS and stained as with antibodies to choline acetyltransferase (Novus Biologicals, goat IgG) and cleaved-caspase 3 (Cell Signaling Technology, Rabbit, IgG) as described in the methods section for “Immunofluorescence staining and imaging of tissue sections”.

### Transmission Electron Microscopy

Extensor digitorum longus (EDL) muscles were dissected and pinned by proximal and distal tendons to the bottom of a petri-dish coated with silicone rubber. Tissues fixed in 2% Glutaraldehyde (Electron Microscopy Sciences, 16000) and 4% paraformaldehyde (Electron Microscopy Sciences, 15700) in 0.1M sodium cacodylate (Electron Microscopy Sciences, 12300) pH 7.4 for 24 hours. Tissues were then transferred from petri-dishes to microcentrifuge tubes with 1% Osmium tetroxide (Electron Microscopy Sciences, 19100) and were kept at room temperature for 1 hour with gentle rotation. Tissues were washed three times with ultrafiltered water and stained with 1% uranyl acetate, dehydrated in ethanol, and embedded in resin (Electron Microscopy Sciences, 14120), and placed in 65 C oven overnight. Transverse sections were generated using a Leica EM UC7 ultramicrotome at 80 nm, placed within grids, and stained for 40 seconds in 3.5% uranyl acetate in 50% acetone for 45 seconds followed by staining in Sato's lead citrate for 2 minutes. Grids were imaged with JOEL JEM 1400 transmission electron microscope using Gatan Microscopy Suite software. Images were quantified by an individual blinded to experimental conditions in ImageJ.

### Statistical Analysis

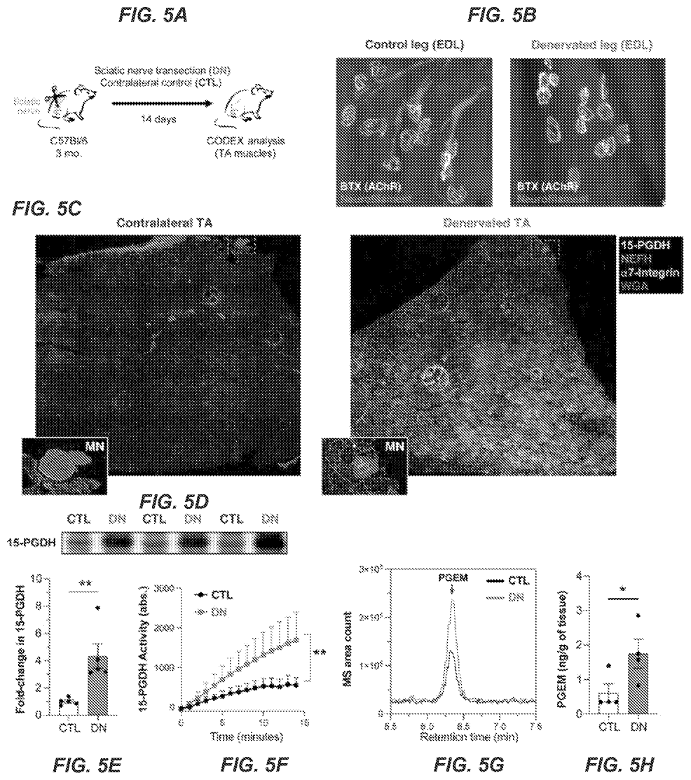

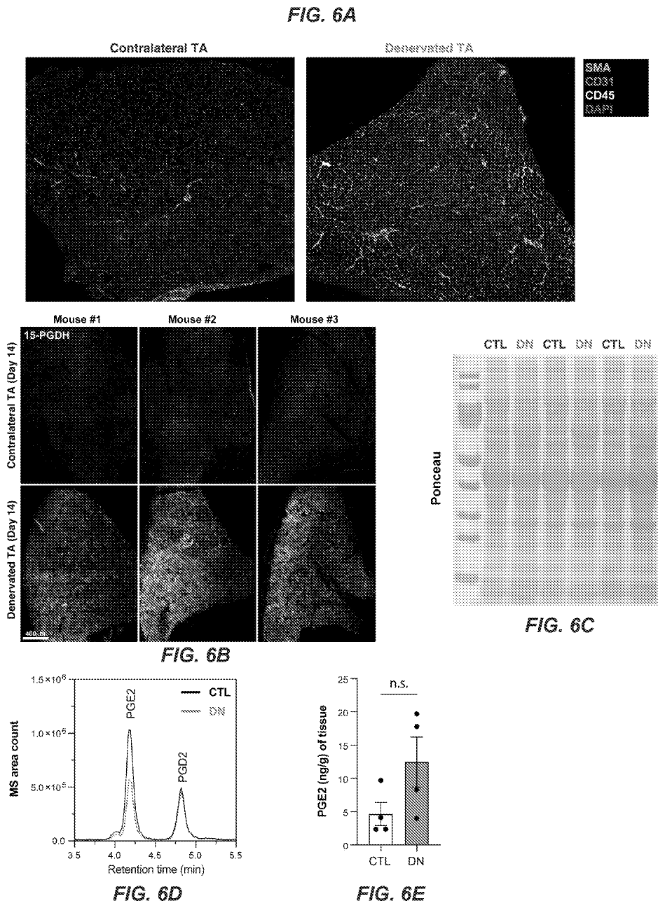

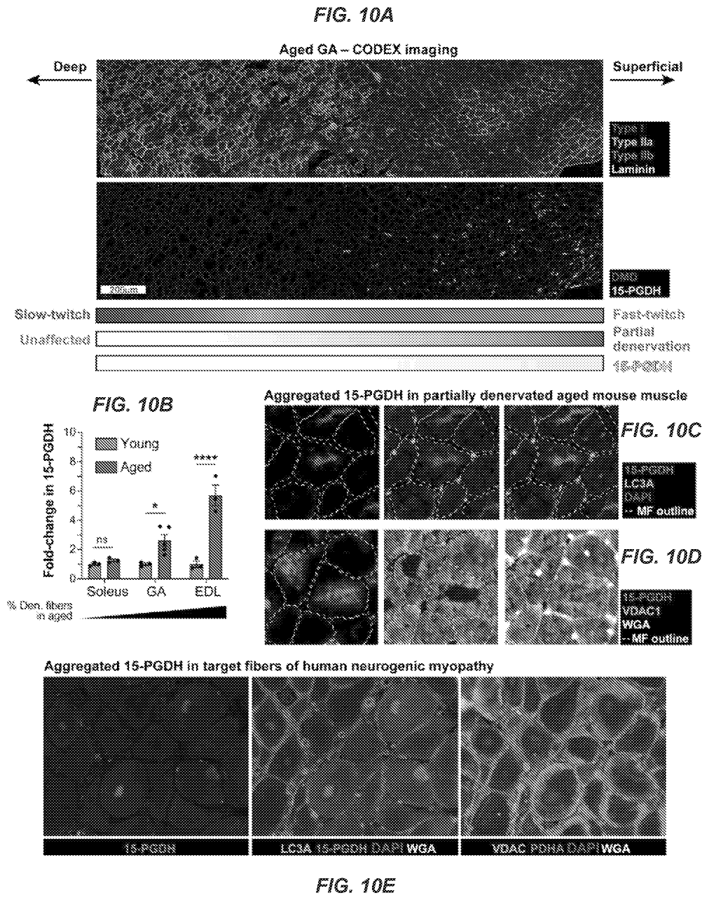

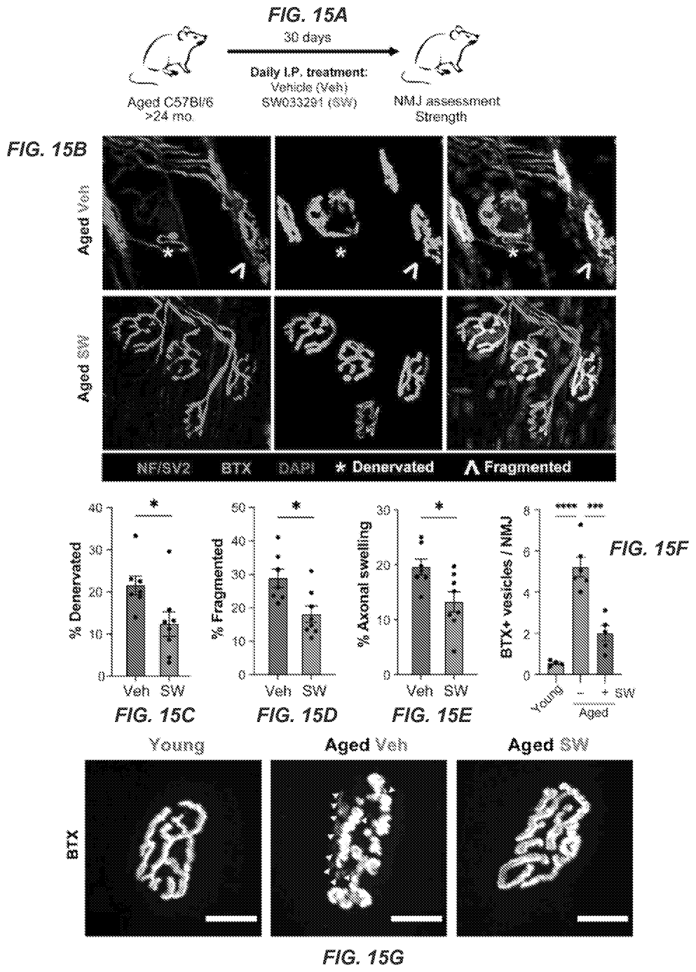

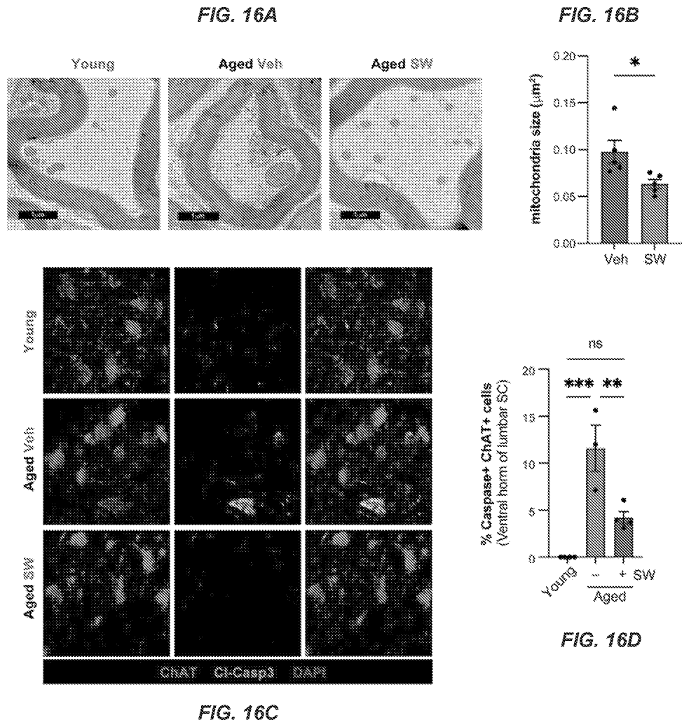

Statistical analysis was performed using GraphPad Prism 9 software. Statistical differences between experimental groups were determined using unpaired t-test. Exceptions to this include the statistical analysis performed in FIG. 5H, and FIG. 6E where paired t-test was used to identify differences between the control and the denervated muscles of mice undergoing unilateral nerve transection, FIG. 2B, FIG. 2D, FIG. 3C, FIG. 15F, FIG. 16D, that one-way ANOVA test was used, and FIG. 10B, and FIG. 1B that two-way ANOVA test was used to identify statistical differences between experimental groups. P<0.05 was considered significant for all statistical tests.

### Antibody List

TABLE 1
List of primary antibodies used in this study

| # | Antibody | Species | Isotype | dilution | | source | Cat. # |
|---|---|---|---|---|---|---|---|
| 1 | 15-PGDH | Mouse | IgG1 | | | Santa Cruz | Sc-271418 |
| | | | | | | Biotechnology | |
| 2 | 2H3 | Mouse | IgG1 | 2-5 | ug/ml | DSHB | 2H3 |
| | (neurofilament) | | | | | | |
| 3 | Anti-PDHA1 | Mouse | IgG1 | 1:200 | | Abcam | Ab110330 |
| 4 | BA-D5 | Mouse | IgG2b | 2 | ug/ml | DSHB | BA-D5 |
| 5 | BF-F3 | Mouse | IgM | 2 | ug/ml | DSHB | BF-F3 |
| 6 | ChAT | Goat | IgG | 1:200 | | Novus Biologicals | NBP1-30052 |
| 7 | Cleaved | Rabbit | IgG | 1:500 | | Cell Signaling | 9664S |
| | Caspase-3 | | | | | Technology | |
| 8 | Dystrophin | Mouse | IgG1 | 1:100 | | Santa Cruz | sc-73592 |
| | | | | | | Biotechnology | |
| 9 | Laminin | Rabbit | Polyclonal | 2 | ug/ml | Abcam | 11575 |
| 10 | LC3A/B | Rabbit | IgG | 1:500 | | Cell Signaling | 12741 |
| | | | | | | Technology | |
| 11 | NCAM1 | Rabbit | Monoclonal | 1:200 | | Abcam | Ab220360 |
| 12 | Neurofilament | Rabbit | polyclonal | 2 | ug/ml | Sigma | N4142 |
| | heavy (NEFH) | | | | | | |
| 13 | P62/SQSTM1 | Guinea | Polyclonal | 1:200 | | Progen | GP62-C |
| | | pig | | | | | |
| 14 | SC-71 | Mouse | IgG1 | 2 | ug/ml | DSHB | SC-71 |
| 15 | SV2 | Mouse | IgG1 | 2-5 | ug/ml | DSHB | SV2 |

| | | | | | | |
|---|---|---|---|---|---|---|
| 16 | VDAC | Rabbit | IgG | 1:200 | Cell Signaling | 4661 |
| | | | | | Technology | |

TABLE 2
List of secondary antibodies and conjugated proteins used in this study

| # | Antibody/protein | Species | Conjugate | dilution | source | Cat. # |
|---|---|---|---|---|---|---|
| 1 | a-bungarotoxin | — | Alexa | 1:500 | ThermoFisher | B35450 |
| | | | Fluor ® 647 | | | |
| 2 | Anti-goat | Bovine | Cy ™3 | 1:500 | Jackson | 805-165-180 |
| | | | | | ImmunoResearch | |
| 3 | Anti-guinea pig | Donkey | Alexa | 1:500 | Jackson | 706-546-148 |
| | | | Fluor ® 488 | | ImmunoResearch | |
| 4 | Anti-mouse IgG γ | Goat | HRP | 1:5000 | Sigma (Chemicon ®) | AP503P |
| | chain specific | | | | | |
| 5 | Anti-mouse IgG1 | Goat | Cy ™3 | 1:500 | Jackson | 115-165-205 |
| | | | | | ImmunoResearch | |
| 6 | Anti-mouse IgG1 | Goat | Alexa | 1:500 | Jackson | 115-545-205 |
| | | | Fluor ® 488 | | ImmunoResearch | |
| 7 | Anti-mouse IgG2b | Goat | DyLight ™ | 1:500 | Jackson | 115-477-187 |
| | | | 405 | | ImmunoResearch | |
| 8 | Anti-mouse IgM | Goat | Cy ™3 | 1:500 | Jackson | 115-165-020 |
| | | | | | ImmunoResearch | |
| 9 | Anti-Rabbit | Goat | Alexa | 1:500 | ThermoFisher | A48228 |
| | | | Fluor ® 488 | | | |
| 10 | Anti-rabbit | Goat | HRP | 1:2500 | Cell Signaling | 7074 |
| | | | | | Technology | |
| 11 | Hoechst 33342 | — | — | 1:1000 | Thermofisher | H3570 |
| 12 | Wheat germ agglutinin | — | Alexa | 1:500 | ThermoFisher | W11261 |
| | | | Fluor ® 488 | | | |

### Reagents and Solutions

TABLE 3
List of reagents used in this study

| # | Name | Source | Cat. # |
|---|---|---|---|
| 1 | Bovine serum albumin | Sigma | A9647 |
| 2 | Butylated hydroxytoluene | Sigma | W218405 |
| 3 | Dextrose | Sigma | D9434 |
| 4 | Kolliphor ® EL (Cremophor ® EL) | Sigma | C5135 |
| 5 | SW033291 | ApexBio | A8709 |
| 6 | Tris-base | Fisher Scientific | BP152-500 |
| 7 | Triton ™ X-100 | Sigma | T8787 |
| 8 | Tween 20 | Sigma | P9416 |

TABLE 4
List of solutions used in this study

| # | Name | Details |
|---|---|---|
| 1 | Blocking solution (IHC) | 5% goat serum, 0.3% Triton-X 100 in PBS |
| 2 | Blocking solution (WB) | 2% Bovine serum albumin in TBST |
| 3 | D5W | 5% (wt/v) dextrose in H₂O (filter sterilized) |
| 4 | PBS-T | Phosphate-buffered saline (PBS, 1X), 0.3% |
| | | Triton-X 100 |
| 5 | TBS-T | 50 mM Tris, 150 mM NaCl, 0.1% (v/v) |
| | | Tween-20 |
| 6 | Vehicle (for in vivo | 85% D5W, 10% ethanol, 5% |
| | drug treatment) | Cremophor EL |

### Results

### 15-PGDH is Upregulated in Denervated Muscle Fibers

15-hydroxyprostaglandin dehydrogenase (15-PGDH) is increased in aging mouse muscles (2). Since motor neuron degeneration and partial denervation of myofibers is a hallmark of skeletal muscle aging (3), the relationship between NMJ dysfunction and 15-PGDH upregulation in aged muscle was investigated.

To directly determine if myofiber denervation leads to 15-PGDH upregulation, unilateral sciatic nerve transection (SNT) in young healthy mice was performed and the myofiber response to denervation was analyzed as described in the method section. FIG. 5A depicts the experimental scheme. CTL indicates control and DN indicates denervated condition. 5 millimeters of the right sciatic nerve was resected at the level of the thigh to ensure complete denervation of the lower limb muscles. Unaffected contralateral legs were used as controls. The extent of denervation was confirmed 14 days post-surgery by immunofluorescence NMJ analysis of whole mount extensor digitorum longus (EDL) muscles. As shown in FIG. 5B on the left panel, innervation of NMJs was confirmed in contralateral EDL muscles by the co-localization of neurofilament at NMJs marked by bungarotoxin that stains acetylcholine receptors (AChRs) on the postsynaptic myofiber sarcolemma. FIG. 5B right panel shows that despite the presence of AChR on the postsynaptic sarcolemma, neurofilament staining was not detected in the denervated EDL muscle, confirming the complete denervation of muscle fibers upon sciatic nerve resection.

Multiplex tissue imaging (CODEX, co-detection by indexing) was used to investigate 15-PGDH levels in denervated muscle and localize its expression to a range of cell types found in skeletal muscle. As shown in FIG. 5C and FIG. 6B, CODEX analysis revealed a significant upregulation of 15-PGDH in the myofibers of denervated tibialis anterior (TA) muscles 14 days post-SNT in young C57BL/6 mice. As shown in FIG. 5C, CODEX further confirmed the efficiency of the SNT through the lack of neurofilament staining in the nerve tracts of TA cross-sections in denervated TAs, compared to robust staining in the axons of motor neurons in the contralateral legs. As shown in FIG. 6A, CODEX analysis further revealed extensive infiltration of immune cells in denervated TAs, at this time point, while vasculature was not affected by SNT. As shown in FIG. 5D, the upregulation of 15-PGDH was quantified by Western blot analysis. FIG. 5E and FIG. 6C depict a ˜4.5-fold increase in 15-PGDH in the GAs of denervated legs after SNT compared to contralateral controls, further supporting the CODEX results.

15-PGDH is the rate-limiting enzyme in the breakdown of PGE2 into 13,14-dihydro-15-keto-PGE2 (PGEM). As shown in FIG. 5G, functional measurement of 15-PGDH specific activity in the protein lysates of denervated and contralateral GA muscles confirmed that 15-PGDH becomes significantly more active upon denervation. To evaluate how the upregulation of 15-PGDH upon denervation impacts prostaglandin metabolism in muscle, LC-MS/MS on contralateral and denervated muscles was performed. This method overcomes the limitations of antibody assays (such as ELISA) cross-reacting with closely related prostaglandins and their metabolites which often share high structural similarities. Notably. LC-MS/MS distinguishes between prostaglandins (PGE2 and PGD2) and PGE2 metabolites, such as PGEM. As shown in FIG. 5H, LC/MS-MS results shows that PGEM, the stable metabolite of PGE2 breakdown, is significantly increased in denervated GA muscles compared to the GAs from the contralateral legs). FIG. 6D and FIG. 6E depict LC/MS-MS results showing that PGE2 and PGD2 levels are unchanged (n.s.).

Together these findings highlight the role of nerve-dependent activity in regulating 15-PGDH expression in myofibers and demonstrate that loss of innervation is also associated with increased PGE2 breakdown via 15-PGDH upregulation.

15-PGDH is Part of a Sustained Gene Program Response after Denervation

Myofiber denervation initiates a series of catabolic molecular programs that lead to atrophy and degradation. Inhibition of 15-PGDH in aged mice had a profound effect in suppressing the muscle expression of atrogenes while boosting metabolic genes. To gain insights into the regulation and biological role of 15-PGDH upregulation upon denervation, longitudinal changes in gene expression were examined upon SNT by RNA-sequencing (5).

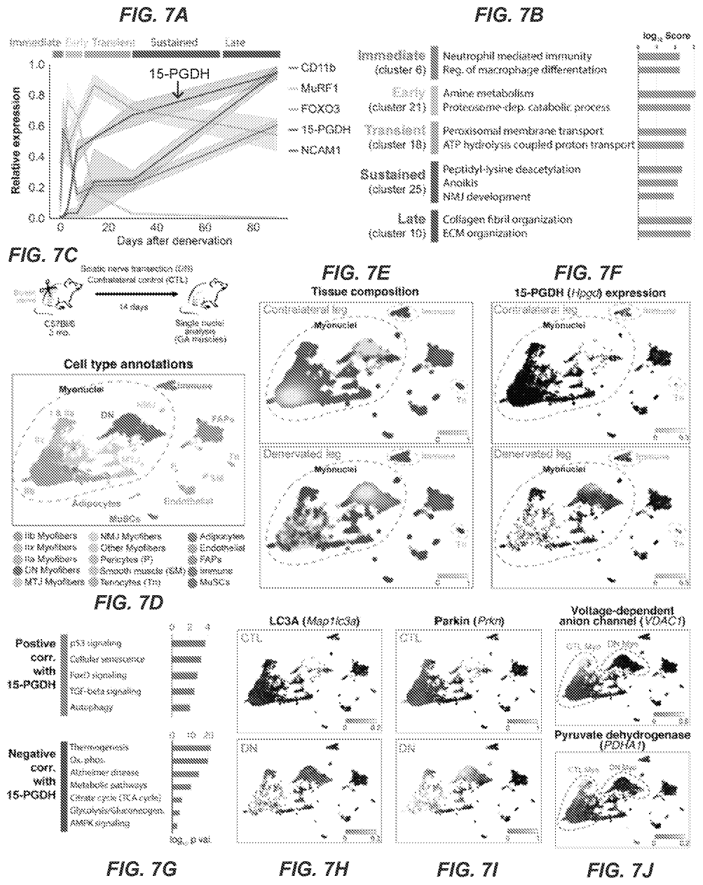

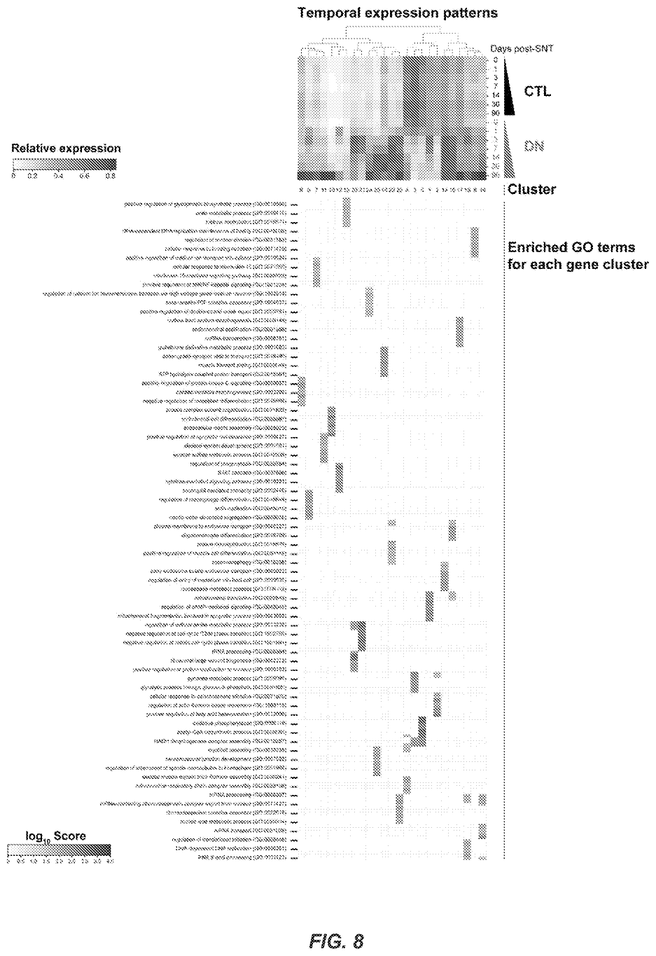

Consistent with results shown at the protein level, as shown in FIG. 7A, line indicated by an arrow, 15-PGDH (Hpgd) mRNA is upregulated as early as day 3 and continues to increase in expression, plateauing around day 21 post-SNT. FIG. 7A also shows that by day 90 post-SNT, 15-PGDH is upregulated ˜50-fold in denervated legs compared to contralateral legs. This temporally regulated expression pattern is distinct from well-known catabolic regulators of denervation including the inflammatory myeloid cell surface marker CD11b (Itgam); muscle RING-finger protein-1, MuRF1 (Trim63); forkhead box protein 03, FOXO3; and neural cell adhesion molecule 1, NCAM1, as shown in FIG. 7A. Indeed, time-course gene set enrichment analysis (GSEA) paired with gene ontology (GO) analysis was performed. As shown in FIG. 8, these denervation genes clustered into gene sets with unique temporal dynamics representing distinct biological processes. FIG. 7A also shows that CD11b shared its immediate and transient response, upregulated in the first 3 days, with other inflammatory genes, enriching for GO terms for neutrophil and macrophage infiltration, which are shown in FIG. 7B. As shown in FIGS. 7A-7B. MuRF1 and other proteosome associated atrogenes were upregulated early for 2 weeks post-SNT, corresponding to the phase of rapid muscle atrophy (5). NCAM1 was grouped with fibrosis genes and ECM proteins that gradually increase in expression toward late stages of the time course, as shown in FIG. 7B, which is consistent with observations of fibrotic build up in muscles with neurogenic myopathies such as SMA.

15-PGDH was grouped into a distinct cluster of genes upregulated after a week post SNT with a sustained expression pattern. GO analysis of this cluster revealed that 15-PGDH is co-expressed with genes associated with peptidyl-lysine deacetylation, anoikis (a form of cell death), and NMJ development. Genes matching the term peptidyl-lysine deacetylation contained histone deacetylase 4 (Hdac4), a class IIa HDAC that functions as a suppressor of hypertrophy and regulates neurogenic muscle atrophy (7, 8). Notably, inhibition of 15-PGDH reduced Hdac4 expression in skeletal muscle of aged mice (2), suggesting that 15-PGDH activity could regulate the expression of other genes in this cluster.

15-PGDH is Associated with Autophagy and Mitophagy in Denervated Myofibers

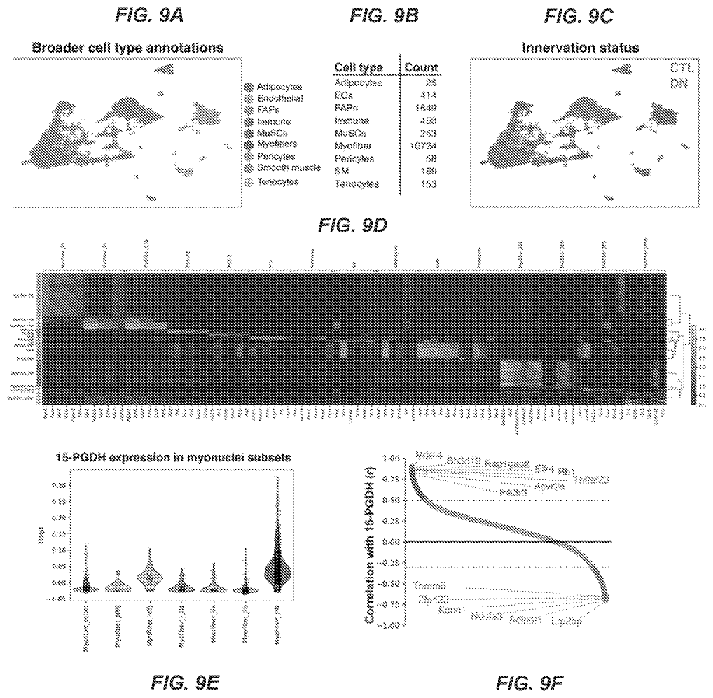

Muscle is composed of a multiplicity of cell types each with distinct molecular responses to denervation. 15-PGDH is expressed by macrophages as well as myofibers in aged muscle (2). Moreover, since myofibers are syncytial cells with known transcriptional heterogeneity in the myonuclei population (2), single-nuclei RNA sequencing (snRNA-seq) was utilized to further resolve the molecular regulation of 15-PGDH. GA muscles from 4 mice undergoing unilateral sciatic nerve transection were collected and processed to obtain intact nuclei. FIG. 7C depicts the experimental scheme of single nuclei RNA sequencing experiment. Nuclei were purified using fluorescence-activated single-cell sorting (FACS) and the 10× Chromium system was used to build libraries for sequencing. FIG. 7D and FIGS. 9A-9D show cell type annotation of single nuclei transcriptome results. 13888 single nuclei transcriptomes from control and denervated muscles were profiled from, which unbiased clustering revealed all major cell types expected in skeletal muscle, including myonuclei subtypes (such as synaptic nuclei and myotendinous junction myonuclei), muscle stem cells (MuSCs), fibroadipogenic progenitors (FAPs), endothelial cells, immune cells, smooth muscle (SM), adipocytes, tenocytes (Tn) and pericytes. As shown in FIG. 7E and FIG. 9C, the most striking cell type composition change in denervated muscle compared to contralateral legs was the loss of type IIb myofibers and the appearance of myonuclei from denervated myofibers clustering into a distinct population. FIG. 7E also shows a subtle increase in the abundance of immune cells upon denervation, in agreement with observations in CODEX results as shown in FIG. 6A.

In contralateral GA muscles, as shown in FIG. 7F, 15-PGDH is expressed by immune cells and in tenocytes but not in myonuclei. By contrast, 15-PGDH becomes highly expressed in the denervated myonuclei cluster in the denervated leg, which are shown in FIG. 7F and FIG. 9E. To further resolve the function of 15-PGDH in denervated myofibers, single cell gene correlation analysis was performed for co-regulated genes match the expression pattern of 15-PGDH. FIG. 9F shows results from single cell gene correlation result with 15-PGDH. As shown in FIG. 7G, positively correlated genes to 15-PGDH were enriched for p53 signaling, cellular senescence, FoxO signaling, TGFbeta signaling, and autophagy; whereas negatively correlated genes were metabolic and encode for mitochondrial enzymes. Interestingly, autophagy genes that correlate with 15-PGDH include LC3A (Map11c3a) and the mitophagy regulator Parkin (Prkn), which are shown in FIGS. 7H-7I, respectively. Both are highly upregulated in the denervated myonuclei cluster and correspond with the reduced of expression mitochondrial membrane marker Voltage-dependent anion channel (VDAC1) and mitochondrial glycolytic enzyme pyruvate dehydrogenase (PDHA1), which are shown in FIG. 7J.

Together these findings demonstrate that myofibers are the main source of 15-PGDH in denervated muscle and establish denervation as a driver of 15-PGDH upregulation in myonuclei. Additionally, these data provide evidence that 15-PGDH correlates with atrophy-associated genes and anti-correlates with metabolic activity genes in denervated myofibers.

### 15-PGDH Aggregates in Denervated Aged Myofibers and Human Myopathies

Given the results in denervated muscles after SNT, the link between 15-PGDH upregulation and neuropathological alterations in aged muscle was investigated. Motor neurons exhibit a selective vulnerability to age with fast-fatigable neurons being the most susceptible while slow motor units tend to be spared with disease progression (10, 11). This is reflected in muscle as fast glycolytic type IIb myofibers, and muscle groups that contain predominantly type IIb myofibers, experience higher rates of denervation than oxidative type I and IIa myofibers. Thus, the expression of 15-PGDH will vary according to the fiber type composition and denervation status of muscles.

First, CODEX was used to investigate the spatial localization of 15-PGDH accumulation in aged GA muscle, a muscle that experiences heterogeneous denervation. FIG. 10A, upper panel, shows denervation of the GA occurs along the skin-to-bone axis (10), aligned with distinct fiber type compositions of glycolytic type IIb myofibers in superficial regions close to the skin and oxidative type I and IIa myofibers in deeper regions close to the bone. Further, as shown in FIG. 10A, while 15-PGDH was absent in young GA (2), 15-PGDH expression was restricted to the type IIb myofibers in superficial regions of the aged GA and excluded from oxidative myofiber types in deep regions, which corroborates a relationship with aging-induced denervation in fast motor units.

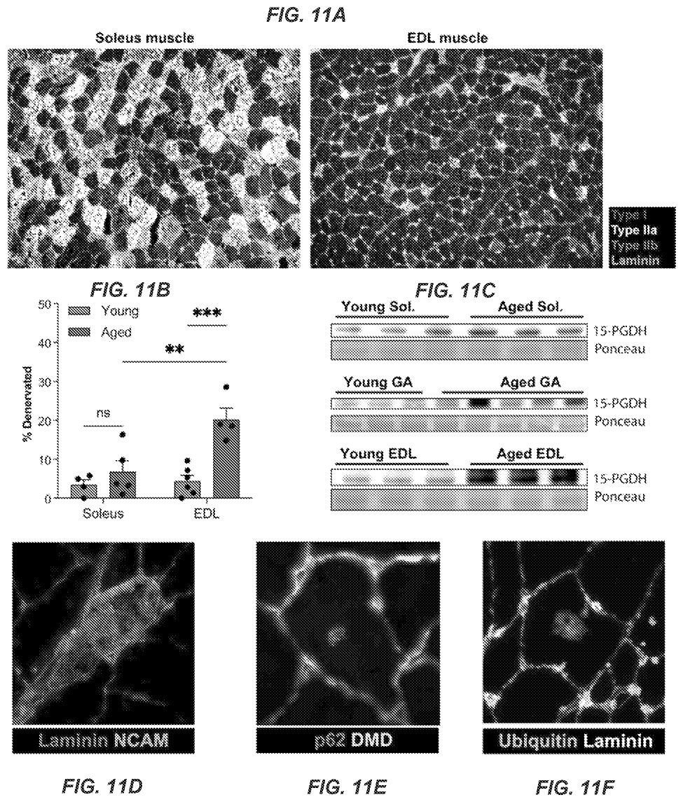

To further confirm the relationship between denervation and 15-PGDH expression in aged muscles, muscle groups that experience different rates of denervation were compared. Denervation and 15-PGDH levels in the mostly fast-twitch extensor digitorum longus (EDL) muscle were measured and compared to mostly slow-twitch soleus muscle of young and aged mice, and results are shown in FIG. 11A. As shown in FIG. 11B, 20% of myofibers in the EDL muscle lacked innervation (no neurofilament staining at postsynaptic AChRs) compared to 3% in young samples, by contrast, aged soleus myofibers do not experience significant denervation. FIG. 10B and FIG. 11C show quantification of 15-PGDH in these muscles by western blot, which revealed a positive correlation between the rate of denervation with aging and the upregulation of 15-PGDH. As shown in FIG. 10B, in aged EDL muscles, the most severely denervated muscle group, 15-PGDH is upregulated more than 5-fold compared to young. These results corroborate with the increase in 15-PGDH upon SNT and suggest that neuropathic alterations drive 15-PGDH upregulation in aging muscle.

A striking feature of 15-PGDH upregulation in aged muscle is its subcellular localization in centralized aggregates within myofibers (2). Given the identification of LC3A and Parkin as 15-PGDH correlated genes by single nuclei analysis, these denervation markers were determined regarding the association with 15-PGDH aggregates in aged muscles. As shown in FIGS. 11D-11F, myofibers with central aggregates also express the denervation marker NCAM1 and have an accumulation of the autophagosome marker p62 and ubiquitin within the aggregates, consistent with denervated myofibers undergoing autophagy-mediated degradation. Moreover, as shown in FIGS. 10C-10D, a strong co-localization of LC3A and 15-PGDH in myofibers and an anticorrelation of the mitochondrial membrane marker VDAC1 with 15-PGDH were observed, suggesting the accumulation of autophagosomes and destruction of mitochondrion within this 15-PGDH+ compartment.

Localized, central accumulation of LC3A and loss of mitochondrion also occurs in human neurogenic myopathies via the development of target, or targetoid fibers. The presence of target fibers is a clinical indication of muscles undergoing denervation with signs of reinnervation. Patient biopsies with clinical diagnosis of target fibers were obtained and immunofluorescence was performed to detect 15-PGDH, LC3A, and mitochondrial proteins VDAC1 and pyruvate dehydrogenase (PDHA). Similar to aged mouse muscle, FIG. 10E shows that 15-PGDH and LC3A were found to co-localize in the “bullseye” region of affected target fibers. Additionally, as shown in FIG. 10E, mitochondria were excluded from the 15-PGDH+ regions as seen by the lack of VDAC1 and PDHA staining.

Collectively, these findings confirm that 15-PGDH is upregulated in a subset of fast glycolytic myofibers experiencing denervation in aged mouse muscle. Its subcellular aggregation co-localizes with the autophagosome in regions lacking mitochondrion. Importantly, this mechanism is conserved in humans as 15-PGDH aggregates are observed in biopsies from neurogenic myopathies.

15-PGDH Inhibition Accelerates Recovery from Nerve Crush Injury

Neurogenic muscle atrophies constitute a large portion of muscle-wasting diseases and lead to the loss of muscle mass and function as a result of injury or disease of the peripheral nervous system. Motor neurons exhibit a remarkable capacity to regenerate after peripheral nerve damage. However, improvement, enhancement, and/or rejuvenation of muscle mass and function relics on formation of functional NMJs after an injury to support rebuilding muscle mass, function, and mobility. In this regard, muscle metabolic fitness and health can regulate formation of functional synapses in a retrograde manner during development and regeneration. Indeed, muscle mitochondrial biogenesis can remodel presynaptic neurons.

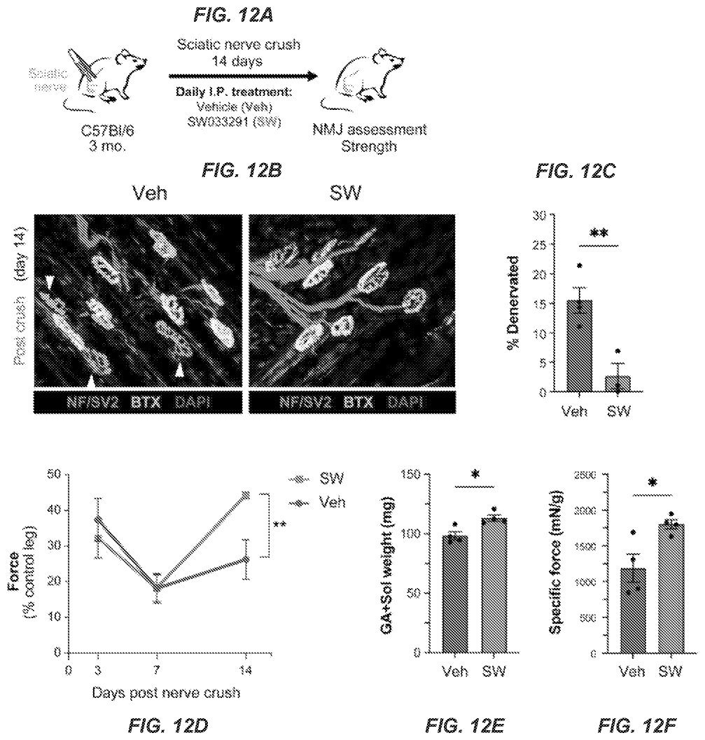

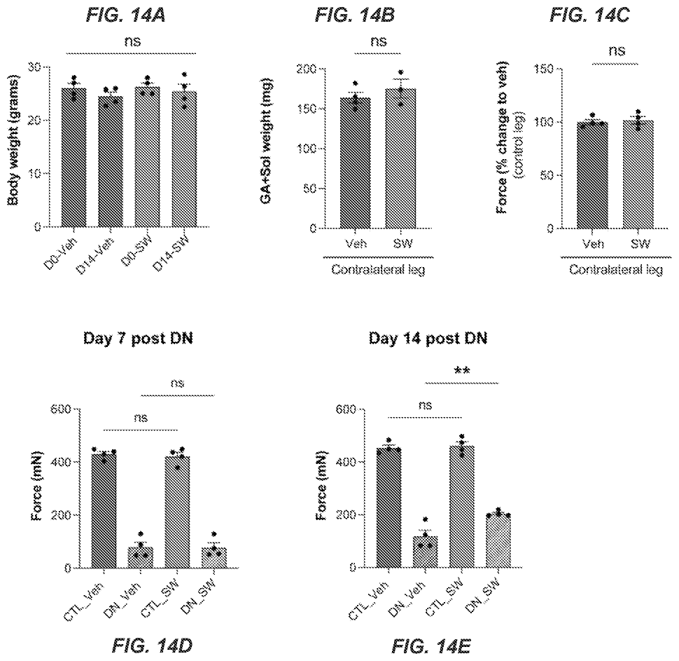

To determine if 15-PGDH regulates NMJ synapse formation after a peripheral nerve injury, unilateral sciatic nerve crush in young healthy mice was performed, and NMJ formation was probed at 14 days post injury (dpi). A partial recovery and re-innervation is expected at this time point. FIG. 12A depicts experimental scheme, showing that injured mice were treated daily with SW or vehicle intraperitoneally for 14 dpi FIG. 12A. Using immunofluorescence analysis of EDL muscles to visualize NMJs, FIGS. 12B-12C show that pharmacological inhibition of 15-PGDH significantly reduced denervation rates in EDLs ipsilateral to the nerve crush compared to vehicle-treated controls 14 dpi. To determine whether the accelerated NMJ formation led to functional recovery, plantar flexor tetanic force at different time points post-injury was measured. To ensure that the measured force is in response to nerve excitation and not direct electrical activation of myofibers, electrodes were located at the level of the tibial nerve and the current was adjusted to a threshold adequate for nerve activation but not muscle fiber depolarization. As shown in FIG. 12D and FIG. 14D, sciatic nerve crush led to a significant decrease in the tetanic plantar flexor force in the injured leg compared to the contralateral (uninjured) leg as early as 3 dpi, which persisted until 7 dpi with no differences between the experimental groups (vehicle and SW). FIG. 12D shows that SW treatment (15-PGDH inhibition) led to a significant increase (69±8% mean±S.D.) in plantar flexion force generation in injured legs while no significant differences were observed in the contralateral legs, as shown in FIGS. 14C-14E. To distinguish the role of muscle mass against neuromuscular function in the increased plantar flexion force, the mass of soleus and gastrocnemius muscles, which are the primary muscles responsible for plantar flexion, were measured. As shown in FIGS. 14A-14B, while mice body weight and muscle mass in the uninjured legs were normalized between the treatment groups. FIG. 12E shows that pharmacological inhibition of 15-PGDH promoted a significant increase in muscle mass in injured legs 14 dpi. To distinguish the contribution of neuromuscular function to the muscle functional performance (plantar flexor), the measured force was normalized to the gastrocnemius and soleus weight to determine muscle-specific force (force per unit of mass). As shown in FIG. 12F, mice treated with SW displayed a profound increase in specific force (52±10% mean±S.D.) compared to vehicle-treated control mice, indicating that the increased function is primarily a result of the increased functional NMJ formation, in alignment with results from immunofluorescence analysis of the NMJs.

Together, these data indicate that 15-PGDH inhibition accelerates recovery and improvement, enhancement, and/or rejuvenation of motor function after peripheral nerve injury. Formation of functional NMJs plays a pivotal role in motor recovery after a nerve injury and these data suggest that 15-PGDH plays a pivotal role formation of functional neuromuscular synapses as its pharmacological inhibition promotes the formation of NMJs in a mouse model of peripheral nerve crush.

### Peripheral Nerve Injury Increases Spinal 15-PGDH Activity

Given the accelerated motor recovery in SW-treated mice, the effect of sciatic nerve crush on spinal 15-PGDH levels was evaluated. Young mice underwent unilateral sciatic nerve crush injury and were treated with vehicle or SW intraperitoneally. Spinal cords were harvested 14 dpi.

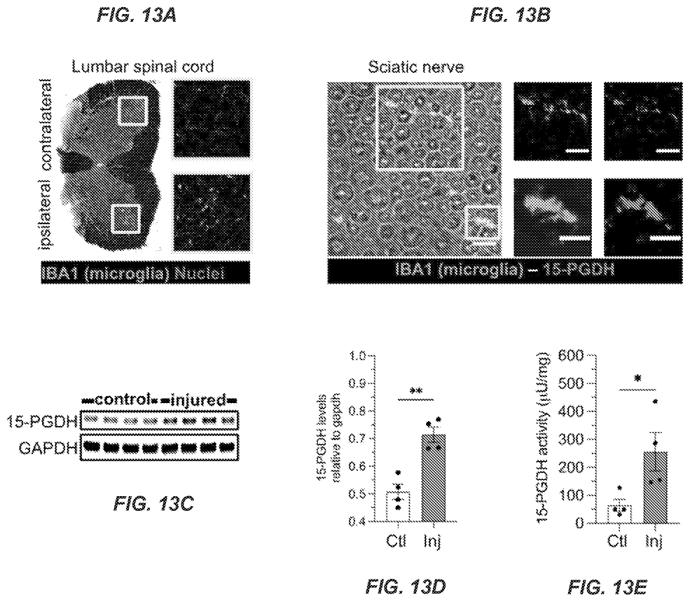

At the transcript level, microglia (macrophages resident in the nervous system), express the highest levels of 15-PGDH among the cells that constitute the spinal cord. These data was corroborated at the protein level by co-immunostaining sciatic nerve cross-sections with the microglia marker Iba-1 and 15-PGDH. As shown in FIG. 13B, 15-PGDH was detected in cells co-stained with Iba-1. This corroborates with the data in muscle, showing tissue-resident macrophages as a major cell type expressing 15-PGDH. Next, to resolve the spatial localization of microglial cells in the spinal cord after peripheral nerve injury, lumbar spinal cord cross-sections of injured mice 14 dpi was stained with Iba-1. As shown in FIG. 13A, peripheral nerve injury causes an increased accumulation of microglia with activated morphology around the motor neurons in the spinal cord ipsilateral to the injury side. In accordance with immunofluorescence results, FIGS. 13C-13D show a significant increase in 15-PGDH protein levels and specific activity in the injured spinal cords compared to uninjured controls. FIG. 13E shows kinetic measurement of 15-PGDH specific activity in lumbar spinal cord of control and injured mice (n=4 each). No differences were observed in animals body weight (FIG. 14A), muscle weight (FIG. 14B), and force (FIGS. 14C-E) of the contralateral control legs between experimental groups (vehicle and SW treatment).

15-PGDH Inhibition Improves, Enhances, and/or Rejuvenates NMJs in Aged Mice

15-PGDH inhibition can increase mitochondrial function and biogenesis in aged mouse muscles (2). Neuromuscular junctions (NMJs) exhibit high morphological plasticity in aging and disease (13, 14). Increased muscle mitochondrial biogenesis remodels NMJs in muscular dystrophies (12, 15). Additionally, misregulation of autophagy and autophagosome formation in muscle fibers leads to precocious denervation and degeneration of postsynaptic AChRs in young mice, phenotypes that are observed in aged muscle. However, the role of increased muscle mitochondrial biogenesis and 15-PGDH inhibition on aging NMJs is not well understood. Additionally, since gene set enrichment and GO analysis grouped 15-PGDH with genes associated with NMJ development and known markers of neurogenic muscle atrophy (HDAC4) in denervated muscle, 15-PGDH inhibition may improve, enhance, and/or rejuvenate age-associated abnormalities that accumulate at the NMJ and lead to the formation of new functional NMJs, as shown in the sciatic nerve crush model. Thus, the effect of pharmacological inhibition of 15-PGDH on morphological status of NMJs in geriatric mice (>26 months) was examined. FIG. 15A shows that Mice were treated once daily intraperitoneally (i.p.) with vehicle or SW033291. EDL muscles were collected after one month of i.p. injections, and FIG. 15B shows immunostained to visualize the presynaptic motor neurons and postsynaptic AChRs. Aged muscles demonstrated a high rate of NMJ-associated alterations such as denervation (21.6±2.3; mean±S.E.M.), axonal swelling (19.6±1.4), and postsynaptic AChR fragmentation (28.9±2.8) in vehicle-treated mice, which are shown in FIGS. 15B-15E and FIG. 18. FIG. 15C-15E shows that 15-PGDH inhibition significantly reduced the incidence of NMJ abnormalities, including denervation (FIG. 15C), AChR fragmentation (FIG. 15D), and motor neuron axonal swelling (FIG. 15E) in aged muscles. Given the morphological improvement, enhancement, and/or rejuvenation of the NMJs, as shown in FIG. 15B, bottom panels, and the lower rate of denervation in aged EDLs after one month of systemic 15-PGDH inhibition (12.4±2.9; mean±S.E.M.), postsynaptic AChRs were evaluated more closely using oil-immersion confocal microscopy. Denervation is correlated with excessive degradation of postsynaptic AChRs in a Trim63-dependant manner in a process that involves autophagy markers LC3A and ubiquitin-binding protein p62 (16-18). 15-PGDH inhibition significantly reduces the ubiquitin ligase Trim63 in aged mouse muscles. Given the finding that 15-PGDH spatially colocalizes with p62 and LC3A in aged muscle fibers, 15-PGDH inhibition impacts AChR structure and stability in aged NMJs were determined. As shown in FIGS. 15F and 15G middle panel, an increased occurrence of AChR-rich endo/lysosomal vesicles in aged NMJs compared to those in young mice were observed. Results from FIGS. 15F-15G shows that 15-PGDH inhibition led to a striking reduction in the number of AChR-rich endo/lysosomal vesicles in aged NMJs, suggesting a improvement, enhancement, and/or rejuvenation of AChR stability in aged muscle. Similarly, aged NMJ morphology was improved, enhanced, and/or rejuvenated to branched and pretzel-like structures found in young EDLs, which are shown in FIG. 15B and FIG. 15G, right panel.

Taken together, these data demonstrate that 15-PGDH inhibition ameliorates morphological changes to the NMJs and improves, enhances, and/or rejuvenates AChR stability in postsynaptic myofibers in aged mouse muscle. These effects are in part due to the impact of 15-PGDH inhibition on promoting myofiber mitochondrial rejuvenation and reduction of muscle atrophy markers such as Trim63 and Hdac4 to improve, enhance, and/or rejuvenate AChR stability and stimulate formation of functional NMJs in a retrograde manner.

### 15-PGDH Inhibition Prevents Motor Neuron Apoptosis in Aged Mice

15-PGDH inhibition reduces the rate of myofiber denervation in aged mice. Next, the health of presynaptic motor neurons in aged mice treated with vehicle and SW. Neuronal PGE2 receptors are positively coupled to cAMP and are shown to elicit neuronal protective effects in vivo in a mouse model of cerebral ischemia. In a mouse model of amyotrophic lateral sclerosis, PGE2 protects motor neurons at physiological concentrations in a cAMP and protein kinase A (PKA) dependent manner. FIG. 16C-D show the health of motor neurons (labeled by staining for choline acetyltransferase, ChAT) by staining for the active form of caspase 3, which plays a central role in cell apoptosis. 15-PGDH inhibition significantly rescues motor neuron cell death (apoptosis) as shown by lower levels of activated caspase-3 in aged lumbar motor neurons.

Given the improved motor neuron health with SW treatment in aged mice, the mitochondrial morphology of heavily myelinated axons in EDL muscle cross-sections were examined by transmission electron microscopy (TEM). FIG. 16A, middle panel, shows analysis of TEM micrographs that with age heavily myelinated axons possess large mitochondria with disorganized morphology. Following one month of SW treatment, aged mitochondria morphology was improved, enhanced, and/or rejuvenated to compact circular mitochondria morphology resembling that seen in young, which are shown in FIGS. 16A-16B). Collectively these data demonstrate that systemic 15-PGDH inhibition exerts a neuroprotective effect on spinal motor neurons in vivo in aged mice.

### Aging is Accompanied by an Increased 15-PGDH Activity in the Spinal Cord

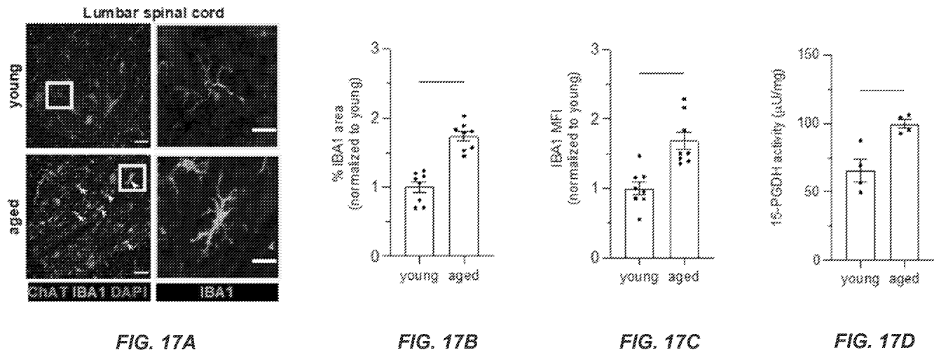

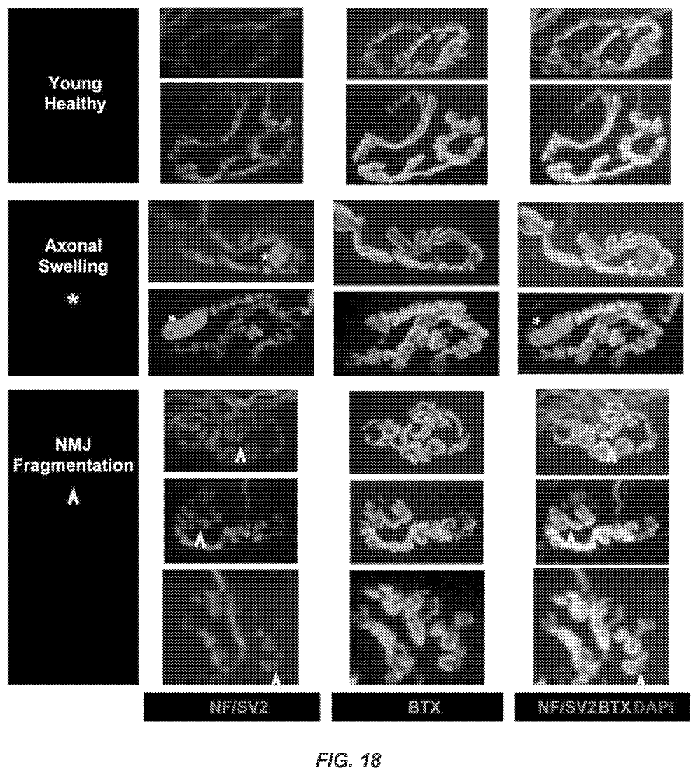

Given the lower rate of NMJ denervation in aged muscle after 15-PGDH inhibition for one month, 15-PGDH activity in the lumbar spinal cord of aged mice was evaluated. FIG. 17D shows that with aging, there is a significant increase in 15-PGDH activity in the lumbar spinal cord with aging. Since microglia express 15-PGDH, to unravel the source of increased 15-PGDH activity, aged lumbar spinal cords were stained with motor neuron marker (ChAT) and IBA1 (microglia). FIGS. 17A-17C show a significant increase in the area covered by IBA1+ cells, and IBA1 immunoreactivity in aged spinal cords. Notably, while microglia demonstrate a ramified morphology in young spinal cord, aged microglia exhibit a drastic change to activated morphology in close proximity of ChAT+ motor neurons. Together, these data show that aging is accompanied by an increase in the spinal 15-PGDH activity.

Aging is accompanied by a drastic loss of muscle mass that affects motor function, mobility, and quality of life in older people (19). There are several factors that contribute to loss of muscle function with age including a functional decline in muscle stem cell (MuSC) capacity to maintain muscle homeostasis (20, 21), changes in tissue extracellular matrix biochemical (22) and biophysical (23, 24) composition, aging-induced changes to muscle inflammatory microenvironment (25-27), pathological remodeling of the NMJs (13, 28), impaired autophagy (29-31) and mitophagy (32), and partial denervation (33, 34). However, relatively little is known about aging muscle fibers and there is a lack of markers to identify muscle fiber health with age. There are currently no therapies for age-related loss of muscle mass (sarcopenia) and gaining a better understanding of myofiber health in aged muscle may help identification of novel therapies to combat sarcopenia and other neurogenic myopathies.

The prostaglandin degrading enzyme, 15-PGDH, accumulates in aged muscle fibers and is associated with reduced muscle PGE2 levels (2). 15-PGDH negatively regulates muscle mass and function in young and aged mice, and its systemic inhibition improves, enhances, and/or rejuvenates muscle function via induction of muscle mitochondrial biogenesis in aging. However, the underlying mechanisms that regulate 15-PGDH or its proteomic interactions in aged muscle fibers were unknown.

As shown in this example, nerve-dependent activity plays a decisive role in regulating the expression of 15-PGDH in muscle fibers. Loss of nerve-dependent activity induces an early and sustained increase in muscular 15-PGDH levels. Single-nuclei RNA sequencing, and CODEX imaging show that myonuclei/muscle fibers exhibit a significant increase in 15-PGDH expression in denervated muscle. RNA-seq data show that congruent with increased 15-PGDH expression, myonuclei downregulate genes correlated with mitochondrial activity and oxidative phosphorylation while, in response to denervation, they upregulate genes correlated with ubiquitin-protein degradation, autophagy, and mitophagy.

Further, as shown herein, analysis of chronically denervated muscle, unravels previously unknown dynamics in cellular responses to muscle neurogenic injury. The gene set enrichment and GO analysis of denervated muscle indicates an immediate infiltration of immune cells post-injury. This is followed by an early catabolic process of protein degradation that declines 14 dpi. Changes in fatty acid metabolism and FOXO3 signaling, which regulates muscle atrophy through the activation of atrogenes (35, 36) exhibit a transient pattern. As shown herein, 15-PGDH shows a significant and sustained upregulation in denervated muscles that coincides with regulators of NMJ development (acetylcholine receptors), autophagy, and histone deacetylase 4, which are known to regulate neurogenic muscle atrophy. On the contrary, genes regulating mitochondrial activity and oxidative phosphorylation are negatively correlated to 15-PGDH gene expression pattern in denervated muscles. These data show 15-PGDH as a part of a sustained genetic program in response to chronic muscle denervation that is associated with markers of neurogenic muscle atrophy such as autophagy, class IIa histone deacetylases, and mitochondrial function.

Given the significant role of partial denervation in aging muscle, the spatial localization of 15-PGDH in aged muscle using CODEX imaging were examined. The gastrocnemius muscle was selected to determine whether 15-PGDH accumulation in aged muscle exhibit a myofiber subtype specificity. Gastrocnemius muscle is composed of deep oxidative (slow) and superficial glycolytic (fast) regions. Results described herein show that 15-PGDH is clustered in fast-glycolytic but not slow and oxidative muscle fibers in aged mouse muscle. These results corroborate with previous research showing the loss of fast motor units in aged lower limb muscles in humans (37) and the selective vulnerability of fast-fatigable neuromuscular synapses in aged mice (11). Therefore, these data suggest that 15-PGDH is a novel marker for aged muscle fibers that experience instances of denervation/reinnervation and partial denervation in the course of aging. Extended age, low-sprouting competence motor neurons abandon a subset of fast-glycolytic myofibers denervated can lead to the accumulation of 15-PGDH in subcellular compartments absent in mitochondrial activity. In contrast, slow-firing motor neurons demonstrate resistance to age-induced decline and thus protect their target muscle fibers from denervation-reinnervation remodeling and subsequent 15-PGDH accumulation with age. These results were confirmed by quantifying denervation rates and 15-PGDH levels in fast extensor digitorum longus (EDL) and slow soleus muscles. These data identify a parallel correlation between denervation rates and 15-PGDH levels in aged mouse muscles. While 15-PGDH shows a significant increase in fast EDLs that experiences over 20% denervation rates with aging, slow-twitch soleus muscles do not exhibit significant changes in either denervation rates or 15-PGDH levels with aging.

Taking advantage of these results using transcriptomic analysis, spatial co-localization of 15-PGDH in aged denervated muscle fibers and muscles of patients with neurogenic myopathies were evaluated. As shown herein, 15-PGDH is localized in subcellular compartments that are low in mitochondrial markers and are enriched in denervation markers such as NCAM (neural cell adhesion molecule) and autophagy markers such as LC3A and ubiquitin-binding protein p62. These results show characteristic of 15-PGDH spatial localization in human neurogenic myopathies.

In addition, a functional role for 15-PGDH in denervated muscles was determined. 15-PGDH plays a pivotal role in hindering repair after a neurogenic injury in a mouse model of sciatic nerve crush. Pharmacological inhibition of 15-PGDH for 14 dpi elicits a significant increase in motor function recovery that is parallel with formation of higher numbers of functional NMJs at this time point. The results in geriatric mice show similar evidence. Pharmacological inhibition of 15-PGDH in geriatric mice promotes formation of new NMJs and ameliorates age-associated abnormalities that accumulate at the neuromuscular junction. Given the striking effect of 15-PGDH in rejuvenating aged muscle mitochondria, and role of muscle retrograde signaling in retaining NMJ health, 15-PGDH remodels aged NMJs via improvement, enhancement, and/or rejuvenation of myofiber metabolic health. These results show that short-term small molecule treatment can accelerate motor recovery following motor nerve injury and elicits formation of functional NMJs in geriatric mice.

Overall, these results show how 15-PGDH is regulated within syncytial myofibers and uncover a role for nerve-dependent activity in regulating 15-PGDH expression in myofibers. Using CODEX imaging, the expression pattern of 15-PGDH in aged mouse gastrocnemius muscle that ties closely with myofiber denervation patterns in aging are shown herein. These data also show that short-term drug intervention partially improves, enhances, and/or rejuvenates aged NMJ morphology and AChR stability. In addition, these results show a role for 15-PGDH inhibition in accelerating motor recovery after nerve crush injury. These results provide strong preclinical data for 15-PGDH inhibitors as potential therapeutic molecules to improve, enhance, and/or rejuvenate motor function in neurogenic myopathies and aging.

### REFERENCES

1. A. T. V. Ho, A. R. Palla, M. R. Blake, N. D. Yucel, Y. X. Wang, K. E. G. Magnusson, C. A. Holbrook, P. E. Kraft, S. L. Delp, H. M. Blau, Prostaglandin E2 is essential for efficacious skeletal muscle stem-cell function, augmenting regeneration and strength. Proc. Natl. Acad. Sci. USA. 114, 6675-6684 (2017).
2. A. R. Palla, M. Ravichandran, Y. X. Wang, L. Alexandrova, A. V. Yang, P. Kraft, C. A. Holbrook, C. M. Schurch, A. T. V. Ho, H. M. Blau, Inhibition of prostaglandin-degrading enzyme 15-PGDH rejuvenates aged muscle mass and strength. Science. 371 (2021), doi:10.1126/science.abc8059.
3. Y. C. Jang, H. Van Remmen, Age-associated alterations of the neuromuscular junction. Experimental Gerontology. 46, 193-198 (2011).
5. J. T. Ehmsen, R. Kawaguchi, R. Mi, G. Coppola, A. Hoke, Longitudinal RNA-Seq analysis of acute and chronic neurogenic skeletal muscle atrophy. Scientific Data. 6, 179 (2019).
7. L. Luo, S. C. Martin, J. Parkington, S. M. Cadena, J. Zhu, C. Ibebunjo, S. Summermatter, N. Londraville, K. Patora-Komisarska, L. Widler, H. Zhai, A.-U. Trendelenburg, D. J. Glass, J. Shi, HDAC4 Controls Muscle Homeostasis through Deacetylation of Myosin Heavy Chain, PGC-1α, and Hsc70. Cell Reports. 29, 749-763.e12 (2019).
8. V. Moresi, A. H. Williams, E. Meadows. J. M. Flynn, M. J. Potthoff. J. McAnally, J. M. Shelton. J. Backs, W. H. Klein. J. A. Richardson, R. Bassel-Duby, E. N. Olson, Myogenin and Class II HDACs Control Neurogenic Muscle Atrophy by Inducing E3 Ubiquitin Ligases. Cell. 143, 35-45 (2010).
10. S. Pun, A. F. Santos. S. Saxena, L. Xu, P. Caroni. Selective vulnerability and pruning of phasic motoncuron axons in motoneuron disease alleviated by CNTF. Nat Neurosci. 9, 408-419 (2006).
11. D. Frey, C. Schneider, L. Xu, J. Borg, W. Spooren, P. Caroni, Early and Selective Loss of Neuromuscular Synapse Subtypes with Low Sprouting Competence in Motoneuron Diseases. J. Neurosci. 20, 2534-2542 (2000).
13. G. Valdez. J. C. Tapia, H. Kang, G. D. Clemenson, F. H. Gage, J. W. Lichtman, J. R. Sanes, Attenuation of age-related changes in mouse neuromuscular synapses by caloric restriction and exercise. PNAS. 107, 14863-14868 (2010).
14. R. M. Grady, H. Teng, M. C. Nichol, J. C. Cunningham, R. S. Wilkinson, J. R. Sanes, Skeletal and Cardiac Myopathies in Mice Lacking Utrophin and Dystrophin: A Model for Duchenne Muscular Dystrophy. Cell. 90, 729-738 (1997).
15. C. Handschin, Y. M. Kobayashi, S. Chin, P. Scale, K. P. Campbell, B. M. Spiegelman, PGC-1α regulates the neuromuscular junction program and ameliorates Duchenne muscular dystrophy. Genes Dev. 21, 770-783 (2007).
16. M. M. Khan, S. Strack, F. Wild, A. Hanashima, A. Gasch, K. Brohm, M. Reischl, S. Carnio, D. Labeit, M. Sandri, S. Labeit, R. Rudolf, Role of autophagy, SQSTM1, SH3GLB1, and TRIM63 in the turnover of nicotinic acetylcholine receptors. Autophagy. 10, 123-136 (2014).
17. R. Rudolf, J. Bogomolovas, S. Strack, K.-R. Choi, M. M. Khan, A. Wagner, K. Brohm, A. Hanashima. A. Gasch, D. Labeit, S. Labeit, Regulation of nicotinic acetylcholine receptor turnover by MuRF1 connects muscle activity to endo/lysosomal and atrophy pathways. AGE. 35, 1663-1674 (2013).
18. F. Wild, M. M. Khan, T. Straka, R. Rudolf. Progress of endocytic CHRN to autophagic degradation is regulated by RAB5-GTPase and T145 phosphorylation of SH3GLB1 at mouse neuromuscular junctions in vivo. Autophagy. 12, 2300-2310 (2016).
19. R. A. Fielding, B. Vellas, W. J. Evans, S. Bhasin, J. E. Morley, A. B. Newman, G. Abellan van Kan, S. Andrieu, J. Bauer, D. Brcuille. T. Cederholm, J. Chandler, C. De Meynard, L. Donini, T. Harris, A. Kannt, F. Keime Guibert, G. Onder, D. Papanicolaou, Y. Rolland. D. Rooks, C. Sieber. E. Souhami, S. Verlaan, M. Zamboni, Sarcopenia: An Undiagnosed Condition in Older Adults. Current Consensus Definition: Prevalence, Etiology, and Consequences. International Working Group on Sarcopenia. Journal of the American Medical Directors Association. 12, 249-256 (2011).
20. B. D. Cosgrove. P. M. Gilbert, E. Porpiglia, F. Mourkioti, S. P. Lee, S. Y. Corbel, M. E. Llewellyn, S. L. Delp, H. M. Blau, Rejuvenation of the muscle stem cell population restores strength to injured aged muscles. Nat Med. 20, 255-264 (2014).
21. A. C. Keefe, J. A. Lawson, S. D. Flygare, Z. D. Fox, M. P. Colasanto, S. J. Mathew, M. Yandell, G. Kardon, Muscle stem cells contribute to myofibres in sedentary adult mice. Nat Commun. 6, 7087 (2015).
22. J. V. Chakkalakal, K. M. Jones, M. A. Basson, A. S. Brack, The aged niche disrupts muscle stem cell quiescence. Nature. 490, 355-360 (2012).
23. C. M. Madl, I. A. Flaig, C. A. Holbrook, Y. X. Wang, H. M. Blau, Biophysical matrix cues from the regenerating niche direct muscle stem cell fate in engineered microenvironments. Biomaterials. 275, 120973 (2021).
24. P. M. Gilbert, K. L. Havenstrite, K. E. G. Magnusson, A. Sacco, N. A. Leonardi, P. Kraft, N. K. Nguyen, S. Thrun, M. P. Lutolf. H. M. Blau, Substrate Elasticity Regulates Skeletal Muscle Stem Cell Self-Renewal in Culture. Science. 329, 1078-1081 (2010).
25. R. S. Blanc, J. G. Kallenbach, J. F. Bachman, A. Mitchell, N. D. Paris, J. V. Chakkalakal, Inhibition of inflammatory CCR2 signaling promotes aged muscle regeneration and strength recovery after injury. Nat Commun. 11, 4167 (2020).
26. D. W. Melton, A. C. Roberts, H. Wang, Z. Sarwar, M. D. Wetzel, J. T. Wells, L. Porter, M. T. Berton, L. M. McManus. P. K. Shireman, Absence of CCR2 results in an inflammaging environment in young mice with age-independent impairments in muscle regeneration. J Leukoc Biol. 100, 1011-1025 (2016).
27. A. Kalinkovich, G. Livshits, Sarcopenic obesity or obese sarcopenia: A cross talk between age-associated adipose tissue and skeletal muscle inflammation as a main mechanism of the pathogenesis. Ageing Res Rev. 35, 200-221 (2017).
28. W. Liu, A. Klose, S. Forman, N. D. Paris, L. Wei-LaPierre, M. Cortés-Lopéz, A. Tan, M. Flaherty, P. Miura, R. T. Dirksen, J. V. Chakkalakal, Loss of adult skeletal muscle stem cells drives age-related neuromuscular junction degeneration. eLife. 6, e26464 (2017).
29. E. Masiero, L. Agatea, C. Mammucari, B. Blaauw, E. Loro, M. Komatsu, D. Metzger, C. Reggiani, S. Schiaffino. M. Sandri, Autophagy Is Required to Maintain Muscle Mass. Cell Metabolism. 10, 507-515 (2009).
30. S. E. Wohlgemuth, A. Y. Seo, E. Marzetti, H. A. Lees, C. Leeuwenburgh, Skeletal muscle autophagy and apoptosis during aging: effects of calorie restriction and life-long exercise. Exp Gerontol. 45, 138-148 (2010).
31. M. Sandri, Autophagy in skeletal muscle. FEBS Letters. 584, 1411-1416 (2010).
32. D. Ryu, L. Mouchiroud, P. A. Andreux. E. Katsyuba, N. Moullan, A. A. Nicolet-dit-Felix. E. G. Williams, P. Jha, G. Lo Sasso, D. Huzard, P. Aebischer, C. Sandi, C. Rinsch, J. Auwerx, Urolithin A induces mitophagy and prolongs lifespan in C. elegans and increases muscle function in rodents. Nat Med. 22, 879-888 (2016).
33. S. Spendiff. M. Vuda, G. Gouspillou, S. Aare, A. Perez, J. A. Morais, R. T. Jagoe, M. Filion, R. Glicksman, S. Kapchinsky, N. J. MacMillan, C. H. Pion, M. Aubertin-Leheudre, S. Hettwer, J. A. Correa, T. Taivassalo, R. T. Hepple, Denervation drives mitochondrial dysfunction in skeletal muscle of octogenarians. J Physiol. 594, 7361-7379 (2016).
34. R. T. Hepple, C. L. Rice, Innervation and neuromuscular control in ageing skeletal muscle. The Journal of Physiology. 594, 1%5-1978 (2016).
35. T. N. Stitt, D. Drujan, B. A. Clarke, F. Panaro, Y. Timofeyva, W. O. Kline, M. Gonzalez, G. D. Yancopoulos, D. J. Glass, The IGF-1/PI3K/Akt Pathway Prevents Expression of Muscle Atrophy-Induced Ubiquitin Ligases by Inhibiting FOXO Transcription Factors. Molecular Cell. 14, 395-403 (2004).
36. G. Milan, V. Romanello, F. Pescatore, A. Armani, J.-H. Paik, L. Frasson, A. Seydel, J. Zhao, R. Abraham, A. L. Goldberg, B. Blaauw, R. A. DePinho, M. Sandri, Regulation of autophagy and the ubiquitin-proteasome system by the FoxO transcriptional network during muscle atrophy. Nature Communications. 6, 6670 (2015).
37. L. J. Einsiedel, A. R. Luff. Alterations in the contractile properties of motor units within the ageing rat medial gastrocnemius. Journal of the Neurological Sciences. 112, 170-177 (1992).
41. A. R. Bauder, T. A. Ferguson, Reproducible Mouse Sciatic Nerve Crush and Subsequent Assessment of Regeneration by Whole Mount Muscle Analysis. JoVE (Journal of Visualized Experiments), e3606 (2012).
42. K. A. Sheth, C. C. Iyer, C. G. Wier, A. E. Crum, A. Bratasz, S. J. Kolb, B. C. Clark, A. H. M. Burghes, W. D. Arnold. Muscle strength and size are associated with motor unit connectivity in aged mice. Neurobiology of Aging. 67, 128-136 (2018).
43. E. L. Mintz, J. A. Passipieri, D. Y. Lovell. G. J. Christ, Applications of In Vivo Functional Testing of the Rat Tibialis Anterior for Evaluating Tissue Engineered Skeletal Muscle Repair. JoVE (Journal of Visualized Experiments), e54487 (2016).

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method of improving, enhancing, and/or rejuvenating neuromuscular junction (NMJ) morphology and/or function, and/or inducing and/or promoting formation of NMJs, in a subject having degeneration of NMJs, the method comprising: administering to the subject an amount of a 15-PGDH inhibitor effective to inhibit 15-PGDH activity and/or reduce 15-PGDH levels in the subject, thereby improving, enhancing, and/or rejuvenating NMJ morphology and/or function, and/or inducing and/or promoting formation of NMJs, in the subject.

2. The method of claim 1, wherein the method results in increased pre-synaptic motor neuron and postsynaptic AChR juxtaposition and/or connectivity.

3. The method of claim 1 or 2, wherein the method results in a decreased number of fragmented acetylcholine receptor (AChR) clusters at the NMJ.

4. The method of any one of the preceding claims, wherein the method results in a decreased number of NMJs lacking the presence of motor neurons.

5. The method of any one of the preceding claims, wherein the method results in decreased blebbing of motor neuron axons.

6. The method of any one of the preceding claims, wherein the method results in decreased apoptosis of motor neurons.

7. The method of any one of the preceding claims, wherein the method results in enhanced NMJ morphology, and/or increased conduction of nerve signals to the muscle.

8. The method of any one of the preceding claims, wherein the method results in a decreased number of AChR-rich vesicles at the NMJ.

9. The method of any one of the preceding claims, wherein the method results in increased expression and/or localization of AChR at the NMJ.

10. The method of any one of the preceding claims, wherein the method results in decreased AChR degradation.

11. The method of any one of the preceding claims, wherein the method results in increased AChR stability.

12. The method of any one the preceding claims, wherein the method results in improved, enhanced, and/or rejuvenated mitochondrial morphology and/or function in motor neurons.

13. The method of any one of the preceding claims, wherein the method results in improved, enhanced, and/or rejuvenated motor neuron synaptic terminals at the NMJ.

14. The method of any one of the preceding claims, wherein the method results in improved, enhanced, and/or rejuvenated skeletal muscle mass and/or neuromuscular function in the subject.

15. The method of any one of the preceding claims, wherein the subject has muscle denervation and/or partial muscle denervation.

16. The method of any one of the preceding claims, wherein the subject has a neurogenic myopathy, an aged-induced loss of muscle mass, a genetic neuromuscular wasting disorder, nerve trauma or injury, muscle trauma or injury, or any combination thereof.

17. The method of claim 16, wherein the genetic neuromuscular wasting disorder is spinal muscular atrophy (SMA), Duchenne muscular dystrophy (DMD), or amyotrophic lateral sclerosis (ALS).

18. The method of any one of the preceding claims, wherein the subject has or has experienced one or more selected from the group consisting of: acute peripheral nerve injury, muscle disuse, myopathy with neurogenic and autoimmune involvement with target fibers or tubular aggregate formation, and vascular myopathy.

19. The method of claim 18, wherein the acute peripheral nerve injury is selected from the group consisting of: contusion injury, compression-decompression injury, nerve cut, botulinum toxicity, injury due to tenotomy, and sports injury.

20. The method of claim 18, wherein the compression-decompression injury is selected from the group consisting of: edema, carpal tunnel syndrome, Baker's cyst, and repetitive task injury.

21. The method of claim 18, wherein the muscle disuse is selected from the group consisting of: immobilization after bone fracture, prolonged bed rest, recovery after surgery, recovery from ventilator, space flight, and sedentary life-style.

22. The method of claim 18, wherein the myopathy with neurogenic and autoimmune involvement with target fibers or tubular aggregate formation is selected from the group consisting of: Duchenne muscular dystrophy, Becker muscular dystrophy, limb girdle muscular dystrophy, central core disease, distal motor axonal neuropathy, multifocal motor neuropathy, amyotrophic lateral sclerosis, spinal muscular atrophy, multiple sclerosis, ataxia, myotonic dystrophy, neurogenic amyloidosis, proximal myopathy with tubular aggregates, rheumatoid arthritis, Sjögren's syndrome, and myasthenia gravis.

23. The method of claim 22, wherein the myasthenia gravis is selected from the group consisting of: congenital myasthenia gravis, episodic myasthenia gravis, and Lambert-Eaton myasthenic syndrome.

24. The method of any one of the preceding claims, wherein the 15-PGDH inhibitor is selected from the group consisting of a small molecule compound, a blocking antibody, a nanobody, and a peptide.

25. The method of any one of the preceding claims, wherein the 15-PGDH inhibitor is selected from the group consisting of: SW033291 and (+)-SW209415.

26. The method of any one of claims 1-24, wherein the 15-PGDH inhibitor is a thiazolidinedione analog with 15-PGDH inhibitory activity.

27. The method of any one of claims 1-23, wherein the 15-PGDH inhibitor is selected from the group consisting of an antisense oligonucleotide, microRNA, siRNA, and shRNA.

28. The method of any one of the preceding claims, wherein the subject is a human.

29. The method of any one of the preceding claims, wherein the subject is less than 30 years of age.

30. The method of any one of claims 1-28, wherein the subject is at least 30 years of age.

31. The method of any one of the preceding claims, wherein the 15-PGDH inhibitor reduces or blocks 15-PGDH expression.

32. The method of any one of the preceding claims, wherein the 15-PGDH inhibitor reduces or blocks enzymatic activity of 15-PGDH.

33. The method of any one of the preceding claims, wherein the method is independent of muscle cell proliferation.

34. The method of any one of the preceding claims, wherein the administering comprises systemic administration or local administration.
